SEQUENCE LISTING (25 sequences)

SEQ ID NO: 1 - protein, 401 aa, Homo sapiens
MRRAPAAERLLELGFPPRCGRQEPPFPLGVTRGWGRWPIQKRREGARPVPFSERSQEDGRGPAARSSGTLWRIRTRLSLCRDPEPPPPLCLLRVSLLCALRAGGRGSRWGEDGARLLLLPPARAAGNGEAEPSGGPSYAGRMLESSGCKALKEGVLEKRSDGLLQLWKKKCCILTEEGLLLIPPKQLQHQQQQQQQQQQQQQQQPGQGPAEPSQPSGPAVASLEPPVKLKELHFSNMKTVDCVERKGKYMYFTVVMAEGKEIDFRCPQDQGWNAEITLQMVQYKNRQAILAVKSTRQKQQHLVQQQPPSQPQPQPQLQPQPQPQPQPQPQPQSQPQPQPQPKPQPQQLHPYPHPHPHPHSHPHSHPHPHPHPHPHQIPHPHPQPHSQPHGHRLLRSTSNSA

SEQ ID NO: 2 - protein, 405 aa, Mus musculus
MRRTPAAERLSELGFPPRRGRQEPPFPLGVTRGWGGWPIEKRREGPRPVPFSERSPEDGREQPAHGSGILWRVRTRLSLCRDPEPPPPPPPLCLLRVSLLCALRAGGRGSRWGEDGAGLLLLPPAGASGSLKAERSSSTPYAGRMLENSGCKALKEGVLEKRSDGLLQLWKKKCCILTEEGLLLIPPKQLQQQQQQQQPGQGTAEPSQPSGPTVASLEPPVKLKELHFSNMKTVDCVERKGKYMYFTVVMTEGKEIDFRCPQDQGWNAEITLQMVQYKNRQAILAVKSTRQKQQHLVQQQPPQTQQIQPQPQPQIQPQPQPQIQPQPQPQPQPQPQPQPQPQPQQLHSYPHPHPHPYSHPHQHPHPHPHPHPHPHPHPYQLQHAHQPLHSQPQGHRLLRSTSNSA

SEQ ID NO: 3 - protein, 638 aa, Homo sapiens
MSALAGEDVWRCPGCGDHIAPSQIWYRTVNETWHGSCFRCSECQDSLTNWYYEKDGKLYCPKDYWGKFGEFCHGCSLLMTGPFMVAGEFKYHPECFACMSCKVIIEDGDAYALVQHATLYCGKCHNEVVLAPMFERLSTESVQEQLPYSVTLISMPATTEGRRGFSVSVESACSNYATTVQVKEVNRMHISPNNRNAIHPGDRILEINGTPVRTLRVEEVEDAISQTSQTLQLLIEHDPVSQRLDQLRLEARLAPHMQNAGHPHALSTLDTKENLEGTLRRRSLRRSNSISKSPGPSSPKEPLLFSRDISRSESLRCSSSYSQQIFRPCDLIHGEVLGKGFFGQAIKVTHKATGKVMVMKELIRCDEETQKTFLTEVKVMRSLDHPNVLKFIGVLYKDKKLNLLTEYIEGGTLKDFLRSMDPFPWQQKVRFAKGIASGMAYLHSMCIIHRDLNSHNCLIKLDKTVVVADFGLSRLIVEERKRAPMEKATTKKRTLRKNDRKKRYTVVGNPYWMAPEMLNGKSYDETVDIFSFGIVLCEIIGQVYADPDCLPRTLDFGLNVKLFWEKFVPTDCPPAFFPLAAICCRLEPESRPAFSKLEDSFEALSLYLGELGIPLPAELEELDHTVSMQYGLTRDSPP

SEQ ID NO: 4 - protein, 451 aa, Mus musculus
MHISPNNRNAIHPGDRILEINGTPVRTLRVEEVEDAIKQTSQTLQLLIEHDPVPQRLDQLRLDARLPPHMQSTGHTLMLSTLDTKENQEGTLRRRSLRRSNSISKSPGPSSPKEPLLLSRDISRSESLRCSSSYSQQIFRPCDLIHGEVLGKGFFGQAIKVTHKATGKVMVMKELIRCDEETQKTFLTEVKVMRSLDHPNVLKFIGVLYKDKKLNLLTEYIEGGTLKDFLRSVDPFPWQQKVRFAKGISSGMAYLHSMCIIHRDLNSHNCLIKLDKTVVVADFGLSRLIVEERKRPPVEKATTKKRTLRKSDRKKRYTVVGNPYWMAPEMLNGKSYDETVDVFSFGIVLCEIIGQVYADPDCLPRTLDFGLNVKLFWEKFVPTDCPPAFFPLAAICCKLEPESRPAFSKLEDSFEALSLFLGELAIPLPAELEDLDHTVSMEYGLTRDSPP

SEQ ID NO: 5 - protein, 403 aa, Homo sapiens [sequence omitted]

SEQ ID NO: 6 - protein, 395 aa, Mus musculus
MDRCKENCVSRPVKTTVPFGPKRVLVTEQIPSQNLGSASSGQAQRVLCPSNSQRVPSQAQKLGAGQKPAPKQLPAASVPRPVSRLNNPQKNEQPAASGNDSEKEQASLQKTEDTKKRQWTLEDFDIGRPLGKGKFGNVYLARERQSKFILALKVLFKTQLEKANVEHQLRREVEIQSHLRHPNILRLYGYFHDATRVYLILEYAPLGTVYRELQKLSKFDEQRTATYITELANALSYCHSKRVIHRDIKPENLLLGSNGELKIADFGWSVHAPSSRRTTMCGTLDYLPPEMIEGRMHDEKVDLWSLGVLCYEFLVGMPPFEAHTYQETYRRISRVEFTFPDFVTEGARDLISRLLKHNASQRLTLAEVLEHPWIKANSSKPPTGHTSKEPTSKSS

SEQ ID NO: 7 - protein, 395 aa, artificial sequence
  mutant Aurora A kinase capable of accepting orthogonal ATP analogs
MDRCKENCVSRPVKTTVPFGPKRVLVTEQIPSQNLGSASSGQAQRVLCPSNSQRVPSQAQKLGAGQKPAPKQLPAASVPRPVSRLNNPQKNEQPAASGNDSEKEQASLQKTEDTKKRQWTLEDFDIGRPLGKGKFGNVYLARERQSKFILALKVLFKTQLEKANVEHQLRREVEIQSHLRHPNIVRLYGYFHDATRVYLIGEYAPLGTVYRELQKLSKFDEQRTATYITELANALSYCHSKRVIHRDIKPENLLLGSNGELKIADFGWSVHAPSSRRTTMCGTLDYLPPEMIEGRMHDEKVDLWSLGVLCYEFLVGMPPFEAHTYQETYRRISRVEFTFPDFVTEGARDLISRLLKHNASQRLTLAEVLEHPWIKANSSKPPTGHTSKEPTSKSS

SEQ ID NO: 8 - protein, 405 aa, artificial sequence
  modified PHLDA sequence with Aurora a kinse sites removed
MRRTPAAERLSELGFPPRRGRQEPPFPLGVTRGWGGWPIEKRREGPRPVPFSERSPEDGREQPAHGSGILWRVRTRLSLCRDPEPPPPPPPLCLLRVALLCALRAGGRGSRWGEDGAGLLLLPPAGASGSLKAERSSSTPYAGRMLENSGCKALKEGVLEKRSDGLLQLWKKKCCILTEEGLLLIPPKQLQQQQQQQQPGQGTAEPSQPSGPTVASLEPPVKLKELHFSNMKTVDCVERKGKYMYFTVVMTEGKEIDFRCPQDQGWNAEITLQMVQYKNRQAILAVKSTRQKQQHLVQQQPPQTQQIQPQPQPQIQPQPQPQIQPQPQPQPQPQPQPQPQPQPQQLHSYPHPHPHPYSHPHQHPHPHPHPHPHPHPHPYQLQHAHQPLHSQPQGHRLLRSTSNSA

SEQ ID NO: 9 - protein, 638 aa, artificial sequence
  modified LIMK2 sequence with Aurora a kinase sites remmoved
MSALAGEDVWRCPGCGDHIAPSQIWYRTVNETWHGSCFRCSECQDSLTNWYYEKDGKLYCPKDYWGKFGEFCHGCSLLMTGPFMVAGEFKYHPECFACMSCKVIIEDGDAYALVQHATLYCGKCHNEVVLAPMFERLSTESVQEQLPYSVTLISMPATTEGRRGFSVSVESACSNYATTVQVKEVNRMHISPNNRNAIHPGDRILEINGTPVRTLRVEEVEDAISQTSQTLQLLIEHDPVSQRLDQLRLEARLAPHMQNAGHPHALSTLDTKENLEGTLRRRALRRSNSISKSPGPSSPKEPLLFSRDISRSESLRCSSSYSQQIFRPCDLIHGEVLGKGFFGQAIKVTHKATGKVMVMKELIRCDEETQKTFLTEVKVMRSLDHPNVLKFIGVLYKDKKLNLLTEYIEGGTLKDFLRSMDPFPWQQKVRFAKGIASGMAYLHSMCIIHRDLNSHNCLIKLDKTVVVADFGLSRLIVEERKRAPMEKATTKKRALRKNDRKKRYAVVGNPYWMAPEMLNGKSYDETVDIFSFGIVLCEIIGQVYADPDCLPRTLDFGLNVKLFWEKFVPTDCPPAFFPLAAICCRLEPESRPAFSKLEDSFEALSLYLGELGIPLPAELEELDHTVSMQYGLTRDSPP

SEQ ID NO: 10 - protein, 747 aa, Homo sapiens
MSQVKSSYSYDAPSDFINFSSLDDEGDTQNIDSWFEEKANLENKLLGKNGTGGLFQGKTPLRKANLQQAIVTPLKPVDNTYYKEAEKENLVEQSIPSNACSSLEVEAAISRKTPAQPQRRSLRLSAQKDLEQKEKHHVKMKAKRCATPVIIDEILPSKKMKVSNNKKKPEEEGSAHQDTAEKNASSPEKAKGRHTVPCMPPAKQKFLKSTEEQELEKSMKMQQEVVEMRKKNEEFKKLALAGIGQPVKKSVSQVTKSVDFHFRTDERIKQHPKNQEEYKEVNFTSELRKHPSSPARVTKGCTIVKPFNLSQGKKRTFDETVSTYVPLAQQVEDFHKRTPNRYHLRSKKDDINLLPSKSSVTKICRDPQTPVLQTKHRARAVTCKSTAELEAEELEKLQQYKFKARELDPRILEGGPILPKKPPVKPPTEPIGFDLEIEKRIQERESKKKTEDEHFEFHSRPCPTKILEDVVGVPEKKVLPITVPKSPAFALKNRIRMPTKEDEEEDEPVVIKAQPVPHYGVPFKPQIPEARTVEICPFSFDSRDKERQLQKEKKIKELQKGEVPKFKALPLPHFDTINLPEKKVKNVTQIEPFCLETDRRGALKAQTWKHQLEEELRQQKEAACFKARPNTVISQEPFVPKKEKKSVAEGLSGSLVQEPFQLATEKRAKERQELEKRMAEVEAQKAQQLEEARLQEEEQKKEELARLRRELVHKANPIRKYQGLEIKSSDQPLTVPVSPKFSTRFHC

SEQ ID NO: 11 - DNA, 58 bp, artificial sequence
  Aurora A primer
ccgggcaccacttggaacagtttatctcgagataaactgttccaagtggtgctttttg

SEQ ID NO: 12 - DNA, 58 bp, artificial sequence
  Aurora A primer
aattcaaaaagcaccacttggaacagtttatctcgagataaactgttccaagtggtgc

SEQ ID NO: 13 - DNA, 58 bp, artificial sequence
  Aurora A primer
ccgggccaatgctcagagaagtactctcgagagtacttctctgagcattggctttttg

SEQ ID NO: 14 - DNA, 58 bp, artificial sequence
  Aurora A primer
aattcaaaaagccaatgctcagagaagtactctcgagagtacttctctgagcattggc

SEQ ID NO: 15 - DNA, 54 bp, artificial sequence
  PHLDA-1 primer
ccgggatggtgcagtacaagaatctcgagattcttgtactgcaccatctttttg

SEQ ID NO: 16 - DNA, 52 bp, artificial sequence
  PHLDA-1 primer
aattcaaaaagatggtgcagtacaagaatctcgagattcttgtactgcacca

SEQ ID NO: 17 - DNA, 54 bp, artificial sequence
  PHLDA-1 primer
ccggtccgcatccacatccacatctcgagatgtggatgtggatgcggatttttg

SEQ ID NO: 18 - DNA, 54 bp, artificial sequence
  PHLDA-1 primer
aattcaaaaatccgcatccacatccacatctcgagatgtggatgtggatgcgga

SEQ ID NO: 19 - protein, 10 aa, Homo sapiens
ALRRASLGAA

SEQ ID NO: 20 - DNA, 54 bp, artificial sequence
  primer for LIMK2
ccggccaactggtactatgagaactcgagttctcatagtaccagttggtttttg

SEQ ID NO: 21 - DNA, 54 bp, artificial sequence
  primer for LIMK2
aattcaaaaaccaactggtactatgagaactcgagttctcatagtaccagttgg

SEQ ID NO: 22 - DNA, 54 bp, artificial sequence
  primer for LIMK2
ccgggctattcacagcagatcttctcgagaagatctgctgtgaatagctttttg

SEQ ID NO: 23 - DNA, 54 bp, artificial sequence
  primer for LIMK2
aattcaaaaagctattcacagcagatcttctcgagaagatctgctgtgaatagc

SEQ ID NO: 24 - DNA, 54 bp, artificial sequence
  primer for LIMK2
ccggcctgctgacagagtacattctcgagaatgtactctgtcagcaggtttttg

SEQ ID NO: 25 - DNA, 54 bp, artificial sequence
  primer for LIMK2
aattcaaaaacctgctgacagagtacattctcgagaatgtactctgtcagcagg

(19) United States
(12) Patent Application Publication
(10) Pub. No.: US 2013/0253037 A1
(43) Pub. Date: Sep. 26, 2013

(54) AURORA A KINASE EFFECTORS

(71) Applicant: Kavita Shah, West Lafayette, IN (US)

(72) Inventor: Kavita Shah, West Lafayette, IN (US)

(73) Assignee: PURDUE RESEARCH FOUNDATION, West Lafayette, IN (US)

(21) Appl. No.: 13/885,170

(22) PCT Filed: Nov. 16, 2011
(86) PCT No.: PCT/US11/60960

§ 371 Date: May 14, 2013

Related U.S. Application Data
(60) Provisional application No. 61/414,169, filed on Nov. 16, 2010.

(51) Int. Cl. C12Q 1/48

(52) U.S. Cl. CPC C12Q 1/485
USPC 514/44 A; 435/15; 435/375

(57) ABSTRACT

Two proteins (PHLDA1 and LIMK2) have been identified as direct targets of Aurora A kinase activity. PHLDA1 downregulation and Aurora A upregulation are strong predictors of poor prognosis for breast cancer patients. In accordance with one embodiment a method of detecting, prognosing and monitoring the presence/progression of cancer, and more specifically breast or prostate cancer, is provided. In one embodiment the method comprises the step of analyzing a biological sample from a patient to detect and/or quantitate the presence of Aurora A, PHLDA1 or LIMK2 amino acid sequences. In one embodiment a method of treating cancer is provided comprising the administration of therapies that enhance the activity of PHLDA1 and/or decrease the activity of LIMK2.

### CROSS REFERENCE TO RELATED APPLICATIONS

This application claims priority under 35 USC §119(e) to U.S. Provisional Application Ser. No. 61/414,169 filed on Nov. 16, 2010, the entire disclosures of which is incorporated herein by reference.

### BACKGROUND

Aurora-A is an oncogenic serine/threonine kinase essential for mitotic spindle assembly, centrosomal separation and maturation. It is activated by phosphorylation and by the microtubule-associated protein TPX2, which also localizes the kinase to spindle microtubules. Aurora A (AA) is one of the genes in the Oncotype Dx assay used for predicting the likelihood of breast cancer recurrence in early-stage, node-negative, estrogen receptor-positive breast cancer, and is overexpressed in a high proportion of pre-invasive and invasive breast carcinomas. Aurora A was the most important gene for predicting breast cancer outcome across multiple datasets in a 3D culture model. In a study of 638 breast cancer patients, high Aurora A expression was strongly associated, even after multivariate analysis, with node status and decreased survival. Polymorphisms in the Aurora A gene are also associated with increased risk of breast cancer and appear to work synergistically with prolonged estrogen exposure. In animal models, Aurora A overexpression induced tumor formation and its inhibition significantly reduced tumor multiplicity and size. The Aurora A gene is also amplified in other types of cancers. Data such as these have resulted in the currently ongoing Phase II clinical trials of several Aurora A inhibitors in advanced solid tumors.

Despite Aurora A's demonstrated potential as a cancer target, the underlying molecular mechanisms of Aurora A-associated malignancy remain elusive. This information is critical for developing pharmacodynamic biomarkers for Aurora A-targeted drugs in clinical trials, developing biomarkers predictive of breast cancer progression and selective targeting of critical malignant effectors of Aurora A independently, or in combination with Aurora A in breast cancer.

In normal cells, Aurora A is expressed during the G2 and M phases of the cell cycle and localizes at the centrosome and mitotic spindle poles. In contrast, in breast tumors, Aurora A is overexpressed in all phases of cell cycle with a diffuse cytoplasmic distribution. Thus, aberrant phosphorylation of cytoplasmic proteins by mislocalized Aurora A is hypothesized to promote malignancy.

More than a dozen Aurora A substrates are known, but few have been identified as potential targets in cancer. With the exception of BRCA1, none are known in breast cancer. Accordingly, there is a need to identify cancer-related targets of Aurora A in breast cancer cells and use them to unravel the mechanisms by which it promotes breast malignancy.

As disclosed herein a chemical genetic approach using an analog-sensitive kinase and orthogonal ATP analog was utilized in a global search of Aurora A substrates. Analog-sensitive kinase is generated by the replacement of a conserved bulky residue (gatekeeper residue) in kinase subdomain V with a glycine (analog-sensitive-1, as1). A complementary substituent on ATP is created by attaching bulky substituents at the N-6 position of ATP (e.g., N⁶-(benzyl) ATP, N⁶-(phenethyl) ATP etc.). Since the ATP analog is not accepted by other wild-type kinases in the cells, this strategy allows for unbiased identification of direct substrates of any kinase in a global environment.

An analog-sensitive mutant of Aurora A (Aurora A-as 1, L201 G-Aurora A) was generated; however, it poorly accepted the orthogonal ATP analogs. This led to the discovery of a novel mutation which renders Aurora A and Aurora B highly sensitive to orthogonal ATP analogs and PPI-derived inhibitors. Using this modified strategy, several Aurora A substrates were identified.

### SUMMARY

Aurora A has been identified as a potential anti-cancer target, however, the underlying molecular mechanisms of Aurora A-associated malignancy remain elusive. In accordance with one embodiment a novel mutant form of Aurora A kinase is provided that renders the kinase amenable to the chemical genetic approach for detecting kinase substrates. In one embodiment a mutation of two residues (LI85V, L201G) of the Aurora A kinase is provided, producing a mutant possessing a hydrophobic cavity that enables it to accept orthogonal ATP analogs and inhibitors. In accordance with one embodiment, a composition comprising a mutant form of Aurora A kinase is provided, wherein the mutant possesses a hydrophobic cavity that enables it to accept orthogonal ATP analogs. In one embodiment the mutant Aurora A kinase comprises the sequence of SEQ ID NO: 7.

In accordance with one embodiment a method of identifying novel Aurora A substrates is provided. The method comprises the steps of contacting a cell lysate with a labeled orthogonal ATP analog and a mutant Aurora A kinase capable of accepting orthogonal ATP analogs and TPX2, and identifying proteins that are the direct targets of the]Aurora A kinase In one embodiment the orthogonal ATP analog is labeled Phenethyl-ATP, and the Aurora A mutant is the double mutant Aurora A (AA-as7; SEQ ID NO: 7). The targeting protein for Xklp2 (TPX2) is a protein that in humans is encoded by the TPX2 gene and is a known activator of Aurora A. In one embodiment the cell lysate is treated with [[γ-³²P] Phenethyl-ATP and Aurora A-as7/TPX2 complex to identify novel Aurora A substrates. Phenethyl ATP is specific for the engineered kinase and is not accepted by wild type kinases present in the cell lysate. In one embodiment the target proteins are separated using 2D gel electrophoresis, isolated and visualized by autoradiography. Further identification of the isolated peptides can be accomplished using standard techniques known to those skilled in the art.

As disclosed herein two proteins Pleckstrin homology-like domain, family A, member 1 (PHLDA1) and the LIM motif-containing protein kinase 2 (LIMK2) have been identified as direct targets of Aurora A kinase activity. PHLDA1 downregulation and Aurora A upregulation are strong predictors of poor prognosis for cancer patients, in breast, cancer. However, they have been not analyzed together. Accordingly, in one embodiment PHLDA1 overexpression may be an alternative way to modulate Aurora A deregulation in breast cancer. Stimulated PHLDA1 overexpression can also be conducted in conjunction with Aurora A inhibition as a means of treating breast or prostate cancer, or other cancers. As used herein the phrase “in conjunction with” is intended to encompass a method where both treatments (Aurora A inhibition or increased PHLDA1 activity) are administered simultaneously, as well as methods where one treatment is administered sequentially after administration of the other. PHLDA1 is upregulated by estrogen. Therefore, in one embodiment estrogen therapy along with Aurora A inhibitors can be used to treat cancer, including for example breast, prostate, colorectal and pancreatic cancer. The combined therapy of stimulating PHLDA1 levels and activity while inhibiting Aurora A levels/activity is anticipated to provide enhanced efficacy in killing cancer cells relative to treatment with only Aurora A inhibition or PHLDA1 overexpression.

In another embodiment, applicants anticipate that LIMK2 and Aurora A upregulation will also be associated with poor prognosis for cancer patients, including for example breast, prostate, ovarian, colorectal and pancreatic cancer. Accordingly, in one embodiment inhibition of LIMK2 activity may be an alternative way to treat breast, prostate, colorectal, ovarian and pancreatic cancers and to modulate Aurora A deregulation in these cancers. Inhibition of LIMK2 activity can also be conducted in conjunction with Aurora A inhibition as a means of treating breast or prostate cancer, or other cancers. As used herein the phrase “in conjunction with” is intended to encompass a method where both treatments (LIMK2 inhibition and Aurora A inhibition) are administered simultaneously as well as methods where one treatment is administered sequentially after administration of the other. The combined therapy is anticipated to provide enhanced efficacy in killing cancer cells relative to treatment with only Aurora A inhibition or LIMK2 inhibition.

Analysis of PHLDA1, LIMK2 and Aurora A levels could supplement standard staging information in primary biopsy samples. In accordance with one embodiment a method of detecting, prognosing and monitoring the presence/progression of cancer, and more specifically breast, prostate, ovarian, colorectal or pancreatic cancer, is provided. In one embodiment the method comprises the step of analyzing a biological sample from a patient to detect and/or quantitate the presence of Aurora A, PHLDA1 or LIMK2 protein levels, or nucleic acid sequences encoding said peptides. Monitoring the relative levels of Aurora A, PHLDA1 and/or LIMK2 protein levels over the course of a therapeutic treatment can be used to indicate the effectiveness of the therapy as well as assist in determining treatment strategy. In accordance with one embodiment a method of treating cancer is provided comprising the administration of combination therapies using both Aurora A, LIMK2 and PHLDA1-targeted drugs.

According to an additional aspect of the present invention there is provided a method of diagnosing predisposition to, or presence of, breast, prostate, ovarian, colorectal or pancreatic cancer in a subject. In one embodiment the method comprises detecting and/or quantitating LIMK2 expression and/or activity in a biological sample obtained from the patient, wherein the level of LIMK2 expression and/or activity in the biological sample is correlated with progression of the cancer in the subject, thereby diagnosing predisposition to, or presence of cancer in the subject. In one embodiment detection of LIMK2 expression and/or activity at a level above a pre-determined threshold of activity in the biological sample, is indicative of cancer in the subject. In accordance with one embodiment a method of treating cancer, including ovarian, colorectal, breast or prostate cancer, is provided wherein the expression or activity of LIMK2 is inhibited or prevented. The present data also supports the development of combination therapies using both Aurora A and LIMK2-targeted drugs.

Accordingly, in one embodiment inhibition of LIMK2 expression or activity may be an alternative way to modulate Aurora A deregulation in breast cancer. Inhibition of LIMK2 expression or activity can also be conducted in conjunction with Aurora A inhibition. As used herein the phrase “in conjunction with” is intended to encompass a method where both treatments (Aurora A inhibition or inhibition of LIMK2 activity) are administered simultaneously as well as methods where one treatment is administered sequentially after administration of the other.

LIMK2 inhibition can be conducted using standard techniques know to those skilled in the art including the use of small molecule inhibitors (see for example Harrison et al. (2009) Novel class of LIM-kinase 2 inhibitors for the treatment of ocular hypertension and associated glaucoma. J Med. Chem. 2009 Nov. 12; 52(21):6515-8, the disclosure of which is incorporated herein) and nucleic acid ablation techniques (using RNAi, DNAzyme etc). Therefore, in one embodiment inhibition of LIMK2 expression or activity along with the administration of Aurora A inhibitors can be used to treat cancer, including for example breast, prostate, colorectal, ovarian and pancreatic cancer to provide enhanced efficacy in killing cancer cells. In a further embodiment, a method of treating cancer is provided wherein LIMK2 activity is inhibited in conjunction with PHLDA1 overexpression. In another embodiment, a method of treating cancer is provided wherein LIMK2 activity is inhibited in conjunction with Aurora A activity inhibition and in conjunction with PHLDA1 overexpression. Inhibition of LIMK2 and/or Aurora A activity can be conducted using any known technique including for example nucleic acid approaches as well as small molecule inhibitors or any combination thereof. Similarly, PHLDA1 overexpression can also be conducted using any known techniques including for example genetic and chemical approaches and any combination thereof.

### BRIEF DESCRIPTION OF THE DRAWINGS

FIGS. 1A-1C: Chemical genetic screen reveals PHLDA1 as a direct substrate of Aurora A. FIG. 1A is a photograph of an SDS-PAGE gel showing the results obtained when 6-His-PHLDA1 was incubated with [γ-32P]ATP in kinase buffer for 15 minutes either alone (lane 3), or with 6-His-Aurora-A and 6-His-TPX2 (lane 2) as described in the Materials and Methods of Example 1. Lane 1 shows Aurora A and TPX2 with [γ-32P]ATP, but without PHLDA1. The data demonstrates that PHLDA1 is directly phosphorylated by Aurora A. FIG. 1B is a photograph of an SDS-PAGE gel showing the results obtained when PHLDA1 was immunoprecipitated from MDA-MB-231 cells, and Aurora A binding analyzed (lane 2). Aurora A and IgG immunoprecipitates were used as positive and negative controls respectively (lanes 1 and 3). The results demonstrate that Aurora A and PHLDA1 associate in MDAMB-231 cells. FIG. 1C is a photograph of an SDS-PAGE gel showing the results obtained when Aurora A was immunoprecipitated from MDA-MB-231 cells, and PHLDA1 binding analyzed (lane 2). PHLDA1 and IgG immunoprecipitates were used as positive and negative controls respectively (lanes 1 and 3).

FIGS. 2A-2D: Aurora A negatively regulates PHLDA1 protein levels. FIG. 2A is a Western blot demonstrating that Aurora A ablation upregulates PHLDA1 in MDA-MB-231 cells. MDA-MB-231 cells were transfected with scrambled shRNA (lane 1), Aurora-A-specific shRNA1 (lane 2) and Aurora A shRNA2 (lane 3) and Aurora A and PHLDA1 levels analyzed after 30 hours. β-actin was used as loading control. FIG. 2B is a Western blot demonstrating that Aurora A overexpression decreases PHLDA1 levels. Wild-type HA-tagged Aurora A-MDA and mutant AA-as7-MDA cells were generated by infecting the corresponding retrovirus, followed by puromycin selection. Aurora A and PHLDA1 levels were analyzed in MDA-MB-231, Aurora A-MDA cells and Aurora A-as7-MDA cells, using β-actin as a control. FIG. 2C presents data demonstrating that Aurora A phosphorylates PHLDA1 at Ser 98. 6-His-tagged wild-type PHLDA1, (S78A) PHLDA1 and (S98A) PHLDA1 were phosphorylated using Aurora A, TPX2 and [γ-32P]ATP for 15 minutes. FIG. 2D further presents data demonstrating Aurora A promotes PHLDA1 degradation by phosphorylating S98. 6-His-tagged wild-type PHLDA1 or (S98A) PHLDA1 was transfected into Aurora A-MDA cells. After 30 hours, Aurora A and PHLDA1 levels were analyzed.

FIG. 3A-3D: PHLDA1 negatively regulates Aurora A protein levels. FIG. 3A is a Western blot demonstrating that PHLDA1 overexpression decreases Aurora A levels. PHLDA1-MDA cells were generated by infecting the cells with a retrovirus expressing the desired gene, followed by puromycin selection. Aurora A and PHLDA1 levels were analyzed in MDA-MB-231 and PHLDA1-MDA cells, using actin as control. FIG. 3B is a Western blot demonstrating that PHLDA1 ablation upregulates Aurora A in MDA cells. MDA-MB-231 cells were transfected with scrambled shRNA (lane 1), PHLDA1-specific shRNA1 (lane 2) and PHLDA1 shRNA2 (lane 3) and Aurora A and PHLDA1 levels analyzed after 30 hours. Actin was used as loading control. FIG. 3C presents data demonstrating that PHLDA1 overexpression increases Aurora A ubiquitylation. MDA-MB-231 cells were cotransfected with PHLDA1 along with 6-His-Ubiquitin. After 36 hours, MG132 was added (10 μM) for an additional 12 hours. Ubiquitinylated proteins were isolated using Ni-NTA beads. The proteins were separated and analyzed using antibodies against Aurora A and PHLDA1. FIG. 3D presents data demonstrating that Aurora A overexpression increases PHLDA1 ubiquitylation. MDA-MB-231 cells were cotransfected with Aurora A and 6-His-Ubiquitin. Ubiquitylated proteins were isolated, separated and analyzed using antibodies against Aurora A and PHLDA1.

FIGS. 4A & 4B. PHLDA1 is a key oncogenic effector of Aurora A. FIG. 4A is a bar graph demonstrating that Aurora A rescues growth inhibition induced by wild-type PHLDA1, but not (S98A) PHLDA1. MDA-MB-231, PHLDA1-MDA and (S98A) PHLDA1-MDA stable cells were seeded in 12-well plates for 12 hours, followed by Aurora A transfection. After 24 hours, growth rate was measured using an MTT assay. The bar graph shows the mean±s.e.m. *P>0.05. FIG. 4B is a bar graph demonstrating that PHLDA1 overexpression and Aurora A inhibition synergistically promotes cell death. Approximately 2000 cells were seeded per well overnight, followed by incubation with MLN8237 (0.5 μM and 1 μM) or vehicle (DMSO). After 48 hours, cells were analyzed using the MTT assay. A significant decrease in cell death was obtained in cells overexpressing PHLDA1 (*P<0.05 and **P<0.01) in the presence of the Aurora A inhibitor relative to the control.

FIG. 5A-5C: LIMK2 is a novel Aurora A substrate: FIG. 5A is a photo of an SDS PAGE gel demonstrating that LIMK2 is directly phosphorylated by Aurora A. Aurora A/TPX2 complex (on beads) was pre-incubated with 10 μM ATP for 15 min in a kinase buffer for activating Aurora A and reducing background phosphorylation. The beads were washed twice with kinase buffer, and subjected to kinase assay with either [₃₂P] ATP alone (lane 1), or with 6-His-LIMK2 and [₃₂P] ATP (lane 2) for 15 min. Lane 3 shows LIMK2 kinase incubated with [₃₂P] ATP. Phosphorylation is only observed in the presence of the Aurora A/TPX2 complex and the LIMK2 kinase. FIG. 5B is a Western blot demonstrating that Aurora A and LIMK2 associate in MDA-MB-231 cells. LIMK2 was immunoprecipitated from MDA-MB-231 cells, and Aurora A binding analyzed (lane 3) using antibody specific for Aurora A. Aurora A (lane 2) and IgG IP (lane 1) were used as positive and negative controls respectively. Densitometric analysis shows that 88% of total Aurora A associates with LIMK2. FIG. 5C Aurora A and LIMK2 associate in MDA-MB-231 cells. Aurora A was immunoprecipitated from MDA-MB-231 cells, and LIMK2 binding analyzed (lane 2). IgG IP (lane 1) and LIMK2 (lane 3) were used as negative and positive controls respectively. Densitometric analysis shows that 71% of LIMK2 associates with AA.

FIG. 6A-6I. Aurora A positively regulates LIMK2 protein levels. FIG. 6A is a western blot that shows that Aurora A ablation depletes LIMK2 in MDA cells. MDA cells were transfected with scrambled shRNA (lane 1), AA-specific shRNA1 (lane 2) and AA-shRNA2 (lane 3) and Aurora A and LIMK2 levels analyzed after 30 h. Actin was used as loading control. FIG. 6B is a western blot that shows that Aurora A overexpression increases LIMK2 levels. Wt HA-Aurora A and mutant HA-AA-as7-MDA cells were generated by infecting the corresponding retrovirus. FIG. 6C is a western blot demonstrating that inhibition of Aurora A kinase using 1-NM-PP1 activity reduces LIMK2 levels. AA-MDA and AA-as7-MDA cells were treated with either DMSO or 250 nM 1-NM-PP1 for 12 h and Aurora A and LIMK2 levels analyzed. FIG. 6D is a Western blot demonstrating that Aurora A inhibits LIMK2 degradation. MDA and AA-MDA cells were treated with cycloheximide (10 μM) for 2 h and 4 h and Aurora A and LIMK2 levels analyzed. FIGS. 6E and 6F are graphs depicting the Aurora A and LIMK2 degradation rate, respectively, in AA-MDA and AA-as7-MDA cells, normalized to actin signal. FIG. 6F is a Western blot demonstrating that Aurora A stabilizes LIMK2 by inhibiting its ubiquitination. MDA cells were cotransfected with Aurora A shRNA along with 6-His-Ubiquitin. Ubiquitinated proteins were isolated and analyzed as described in Materials and Methods of Example 1. FIG. 6H is a Western blot demonstrating that Aurora A-mediated phosphorylation of LIMK2 increases its kinase activity. All kinase assays were conducted for 10 min at RT. 6-His-cofilin (2 μg) was treated with Aurora A/TPX2 and [₃₂P] ATP in a kinase buffer (lane 1). For lane 2,6-His LIMK2 (on beads) was pre-incubated with Aurora A/TPX2 for 15 min in the presence of 10 μM ATP. Beads were washed to remove Aurora A/TPX2 and cold ATP. Phosphorylated LIMK2 was used to phosphorylate cofilin in the presence of [₃₂P] ATP. Lane 3 includes LIMK2, cofilin and [₃₂P] ATP. Lane 4 shows cofilin alone. Proteins were analyzed by Ponceau stain (for cofilin loading), autorad (for determining cofilin phosphorylation), LIMK2 and Aurora A antibodies. FIG. 6I is a Western blot demonstrating that Aurora A increases LIMK2 activity using its kinase activity. Wild type and dominant negative Aurora A (in solution) were used to activate 6-His-LIMK2 (on bead) using 10 μM ATP at 30° C. for 30 minutes. The beads were washed twice with kinase buffer to remove Aurora A, and then incubated with 6-His-cofilin and [₃₂P] ATP at 30° C. for 10 minutes.

FIGS. 7A-7F. LIMK2 positively regulates Aurora A. FIG. 7A is a Western blot demonstrating that LIMK2 ablation downregulates Aurora A. MDA cells were transfected with scrambled shRNA (lane 1), LIMK2-shRNA1 (lane 2) and LIMK2-shRNA2 (lane 3) and Aurora A and LIMK2 levels analyzed after 30 h. FIG. 7B is a Western blot of Aurora A and LIMK2 levels in MDA and LIMK2-MDA cells, demonstrating that LIMK2 overexpression positively regulates Aurora A levels. FIG. 7C is a Western blot demonstrating that LIMK2 stabilizes Aurora A levels. MDA and LIMK2-MDA cells were treated with cycloheximide for 2 and 4 h and Aurora A and LIMK2 levels analyzed. FIGS. 5D and 5E provide graphical representation of Aurora A and LIMK2 degradation rate, respectively, with LIMK2 signal normalized to actin signal. LIMK2 half-life is ˜2 h. FIG. 7F is a Western blot demonstrating that LIMK2 depletion increases Aurora A ubiquitination. MDA cells were co-transfected with LIMK2 shRNA and 6-His-Ubiquitin for 36 h as described in Materials and Methods of Example 2.

FIGS. 8A-8D. Aurora A phosphorylates LIMK2 at 5283, T494 and T505 which increases its protein stability. FIG. 8A is a autoradiograph of an SDS PAGE gel demonstrating that Aurora A phosphorylates LIMK2 at S283, T494 and T505 in vitro. 6-His-tagged wild type, (S283A) LIMK2, (T494A) LIMK2 and (T505A) LIMK2 mutants were phosphorylated using Aurora A/TPX2 and [³²P] ATP for 15 min and the products were separate by electrophoresis. FIG. 8B is a Western blot demonstrating that Aurora A is responsible for phosphorylation of LIMK2 at S283, T494 and T505 sites in cells. Nucleic acids sequence encoding HA-tagged (S283A) LIMK2, (T494A) LIMK2, and (T505A) LIMK2 were transfected in MDA cells. After 30 h, cells were incubated with 0.5 μM MLN8237 for additional 16 h. Gel shift assay was performed using 10% SDS PAGE. Protein levels were analyzed using HA and actin antibodies. FIG. 8C is a western blot showing Aurora A promotes LIMK2 stability by phosphorylating S283, T494 and T505. Nucleic acids sequences encoding HA-tagged wt LIMK2, (S283A, T494A) LIMK2 (2A) and (S283A, T494A, T505A) LIMK2 (3A) were transfected in MDA cells. After 30 h, protein levels were analyzed using Aurora A, HA and actin antibodies. FIG. 8D is a Western blot demonstrating Aurora A inhibits LIMK2 ubiquitination by phosphorylating S283, T494 and T505 sites. Wild type, double (2A) and triple (3A) mutants of LIMK2 were transfected in MDA cells along with 6-His Ubiquitin and LIMK2 ubiquitination analyzed.

FIGS. 9A & 9B. LIMK2 is not a mitotic target of Aurora A. FIG. 9A is a Western blot demonstrating that LIMK2 expression is not cell cycle regulated. MDA cells arrested at G1/S using double thymidine block were released for varying periods and Aurora A and LIMK2 levels analyzed. D and L refer to darker and lighter exposures of Aurora A IB respectively. FIG. 9B is a Western blot displaying the LIMK2 and Aurora A levels in HCT116 cells following double thymidine release.

FIGS. 10A-10E. LIMK2 is a key oncogenic effector of Aurora A. FIG. 10A is a graph plotting the growth curves of various MDA cells and demonstrating LIMK2 promotes cell proliferation in MDA cells. MDA, AA-MDA, LIMK2-ablated MDA and LIMK2-ablated-AAMDA cells were seeded in 96-well plate and harvested at 6 h, then every 12 h up to 80 h. At the end of incubation, MTT solution was added and absorbance measured. FIG. 10B is a bar graph measuring the growth of various MDA cells and demonstrating LIMK2's role in inhibiting anchorage-independent growth. Soft-agar colony formation assays were performed with MDA, AA-MDA, LIMK2-depleted-MDA and LIMK2-depleted-AA-MDA stable cells. The bar graph shows the mean; error bars, ±SEM (*p>0.05, **p>0.01 as compared to control). Column 1 is control, which includes no cells. FIG. 10C is a bar graph measuring the chemotaxic response of various MDA cells and demonstrating LIMK2 is a potent activator of chemotaxis. The migrating abilities of MDA, AA-MDA, LIMK2-depleted-MDA and LIMK2-depleted-AA MDA cells were determined in a Boyden chamber. The bar graph show the mean; error bars, ±SEM (*p>0.05 as compared to AA-MDA and MDA cells respectively). Grey bars show LIMK2-ablated AA-MDA and LIMK2-ablated-MDA cells. FIG. 10D is a graph plotting the growth curves of MDA and AA-MDA cells in vivo and demonstrating the effect of AA overexpression in MDA cells on subcutaneous tumor growth in female athymic nude mice. Four nude mice were inoculated with MDA cells and AA-MDA cells on mouse right and left shoulders, respectively. The growth of the tumor was monitored and measured every two days. FIG. 10E is a graph plotting the growth curves of LIMK2-ablated AA-MDA and AA-MDA cells in vivo and demonstrating the effect of LIMK2 ablation on subcutaneous tumor growth in athymic nude mice. Four nude mice were inoculated with AA-MDA cells and LIMK2-ablated AA-MDA cells on right and left shoulder respectively. FIG. 10F shows a picture of an athymic nude mouse injected with LIMK2-ablated AA-MDA cells on left shoulder, and AA-MDA on the right shoulder. The pictures were taken 34 days following inoculation.

FIG. 11A-11F Aurora A and LIMK2 positive feedback loop is a common feature in several cancers. FIG. 11A is a Western blot demonstrating that Aurora A ablation in PC3 cells depletes LIMK2. PC3 cells were transfected with scrambled shRNA (lane 1) or Aurora A-shRNA2 (lane 2) and Aurora A and LIMK2 levels analyzed after 30 h. FIG. 11B is a Western blot demonstrating that LIMK2 ablation in PC3 cells depletes Aurora A. PC3 cells were transfected with scrambled shRNA (lane 1) or LIMK2-shRNA1, shRNA-2 and shRNA-3 and Aurora A and LIMK2 levels analyzed after 30 h. FIG. 11C is a western blot showing that Aurora A ablation in HCT116 and PaCa2 cells depletes LIMK2. HCT116 and PaCa2 cells were transfected with scrambled shRNA (lanes 1 and 3) or Aurora A-shRNA2 (lanes 2 and 4) and Aurora A and LIMK2 levels analyzed. FIG. 11D is a Western blot demonstrating that LIMK2 ablation in HCT116 and PaCa2 cells depletes Aurora A. HCT116 and PaCa2 cells were transfected with scrambled shRNA (lane 1) or LIMK2-shRNA2 (lane 2) and Aurora A and LIMK2 levels analyzed after 30 h. FIG. 11E is a western blot showing that LIMK2 acts as a pharmacodynamic biomarker for Aurora A kinase activity. Aurora A was inhibited using 0.5 μM MLN8237 in MDA cells and Aurora A, LIMK2 and actin levels analyzed. FIG. 11F is a bar graph demonstrating that LIMK2 ablation works synergistically with Aurora A inhibition in promoting cell death. MDA cells were incubated with MLN8237 (0.5 μM and 1 μM) or the vehicle (DMSO). After 48 h, cells were analyzed using MTT assay. p*<0.05, when compared to the control.

### DETAILED DESCRIPTION

### Definitions

In describing and claiming the invention, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein the term “Aurora kinase substrate” or “Aurora A kinase substrate” are intended to designate any peptide comprising moiety that can be phosphorylated by a protein kinase comprising the sequence of SEQ ID NO: 5 or SEQ ID NO: 6.

As used herein the term “PHLDA-1” encompasses any amino acid sequence comprising the sequence of SEQ ID NO: 1 or SEQ ID NO: 2, or analogs of SEQ ID NO: 1 or SEQ ID NO: 2 comprising an amino acid sequence having greater than 90% sequence identity with SEQ ID NO: 1 or SEQ ID NO: 2 or an amino acid sequence comprising at least an 12 amino acid fragment of SEQ ID NO: 1 or SEQ ID NO: 2.

As used herein the term “LIMK2” encompasses any amino acid sequence comprising the sequence of SEQ ID NO: 3 or SEQ ID NO: 4, or analogs of SEQ ID NO: 3 or SEQ ID NO: 4 comprising an amino acid sequence having greater than 90% sequence identity with SEQ ID NO: 3 or SEQ ID NO: 4 or an amino acid sequence comprising at least an 12 amino acid fragment of SEQ ID NO: 3 or SEQ ID NO: 4.

As used herein the term “TPX2” encompasses any amino acid sequence comprising the sequence of SEQ ID NO: 3 or SEQ ID NO: 4, or analogs of SEQ ID NO: 3 or SEQ ID NO: 4 comprising an amino acid sequence having greater than 90% sequence identity with SEQ ID NO: 3

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

As used herein the term “inhibitor” when used in the context of a protein kinase (e.g., such as Aurora A) is intended to encompass any compound that causes a decrease in activity of the kinase in vivo or in an in vitro assay. Kinase activity for purposes of the present invention includes phosphorylation of its native substrate and binding to native ligands. In one embodiment the inhibitor is any safe and effective compound that can be administered to a patient to decrease the target kinase activity in vivo.

As used herein an “effective” amount or a “therapeutically effective amount” of an inhibitor refers to a nontoxic but sufficient amount of an inhibitor to provide the desired effect. For example one desired effect would be reducing the levels of the target kinase protein or reducing the efficiency of the kinase to phosphorylate its target substrate. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, the term “treating” includes prophylaxis of the specific disorder or condition, or alleviation of the symptoms associated with a specific disorder or condition and/or preventing or eliminating said symptoms. For example, as used herein the term “treating a tumor” will refer in general to maintaining or reducing the tumor size or eliminating detectable cancer cells from the patient undergoing treatment.

The term, “parenteral” means not through the alimentary canal but by some other route such as subcutaneous, intramuscular, intraspinal, or intravenous.

As used herein an amino acid “substitution” refers to the replacement of one amino acid residue by a different amino acid residue.

As used herein, the term “conservative amino acid substitution” is defined herein as exchanges within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln, cysteic acid and homocysteic acid;

III. Polar, positively charged residues:
His, Arg, Lys; Ornithine (Orn)

IV. Large, aliphatic, nonpolar residues:
Met, Leu, Ile, Val, Cys, Norleucine (Nle), homocysteine

V. Large, aromatic residues:
Phe, Tyr, Trp, acetyl phenylalanine

As used herein, the term “antibody” refers to a polyclonal or monoclonal antibody or a binding fragment thereof such as Fab, F(ab′)₂ and Fv fragments that specifically binds to an antigenic site.

As used herein the term “patient” without further designation is intended to encompass any warm blooded vertebrate domesticated animal (including for example, but not limited to livestock, horses, cats, dogs and other pets) and humans.

As used herein the term “cancer patient” is intended to encompass any patient that at one time was diagnosed with cancer and continues to receive treatments related to their cancer. This includes patients with active cancers, those in remission, and patients who have been subsequently deemed cancer free but continue to receive treatment for their cancer. For example breast cancer or ovarian cancer patients may continue to receive aromatase therapy for their cancers long after cancer cells can no longer be detected in their bodies.

### Embodiments

Aurora A kinase is overexpressed in cancers of many origins, which include both solid tumors and hematological malignancies. Over a dozen Aurora A inhibitors are in advanced clinical trials. Although Aurora A inhibition has shown high efficacy in clinical trials, it is also associated with significant side effects, including neutropenia, somnolence, asthenia and transaminitis, presumably because it is expressed in all dividing cells. In normal cells, Aurora A is essential for centrosome duplication and separation, microtubule kinetochore attachment, spindle checkpoint formation and cytokinesis during mitosis. Aurora A null mice die at the blastocyst stage. These findings suggest that selective inhibition of cancer-specific targets of Aurora A should reduce the toxicity associated with systemic Aurora A inhibition in cancer. However, the underlying molecular mechanisms of Aurora A's malignancy remain elusive, primarily due to the lack of known cancer-specific targets of Aurora A.

In accordance with one embodiment a method of identifying substrates of Aurora A is provided, comprising an in vitro assay for Aurora A activity. The assay can be used to identify previously unknown substrates of Aurora A, or alternatively the assay can be used to identify and measure the efficacy of inhibitors to prevent Aurora A phosphorylation of known natural substrates. In one embodiment a kit is provided for conducting Aurora A kinase reactions, said kit comprising an Aurora A kinase, the microtubule-associated protein TPX2, an Aurora A kinase substrate selected from the group consisting of PHLDA-1 and LIMK2, and various reagents for conducting a kinase reaction. In one embodiment the kit comprises the Aurora A kinase complexed to TPX2, including for example an Aurora A/TPX2 complex linked to beads. The kit may include various containers, e.g., vials, tubes, bottles, and the like. Preferably, the kits will also include instructions for use. The kit may include reagents for conducting the kinase reactions including ATP, optionally both in a labeled from and non-labeled form, as well as buffer solutions for conducting and terminating the reactions. In accordance with one embodiment the TPX2 protein comprises the sequence of SEQ ID NO: 10. In a further embodiment the PHLDA-1 substrate comprises a peptide of SEQ ID NO: 1 or SEQ ID NO: 2 and the LIMK2 substrate comprises a peptide of SEQ ID NO: 3 or SEQ ID NO: 4, or peptide fragments or derivatives of SEQ ID NO: 1-4 that are capable of being phosphorylated by Aurora A. In a further embodiment the kit comprises an Aurora A kinase substrate negative control, wherein the negative control comprises a PHLDA-1 or LIMK2 substrate that has been modified to no longer be a substrate for Aurora A. In one embodiment the negative control comprises an amino acid sequence selected from SEQ ID NO: 8 and SEQ ID NO: 9. In one embodiment the Aurora A kinase comprises the sequence of SEQ ID NO: 5, and the PHLDA-1 or LIMK2 substrate comprise the sequence of SEQ ID NO: 1 and SEQ ID NO: 3, respectively. Alternatively, or in addition to the Aurora A kinase of SEQ ID NO: 5, the kit may comprise a derivative form of Aurora A kinase including the Aurora A kinase comprises the sequence of SEQ ID NO: 7. In a further embodiment the kit comprises an orthogonal ATP analog, including for example an ATP analog comprising a bulky substituents at the N-6 position of ATP (such as N-6-Phenethyl ATP), optionally labeled.

In accordance with one embodiment, a composition comprising a mutant form of Aurora A kinase is provided, wherein the mutant possesses a hydrophobic cavity that enables it to accept orthogonal ATP analogs and inhibitors. In one embodiment a mutation of two residues (LI85V, L201G) of the native Aurora A kinase is provided, producing a mutant (i.e., SEQ ID NO: 7) possessing a hydrophobic cavity that enables it to accept orthogonal ATP analogs and inhibitors.

Applicants have discovered that not only is LIMK2 a substrate for Aurora A, but Aurora A inhibition rapidly degrades LIMK2 (half-life ˜2 h). Accordingly, LIMK2 can serve as a pharmacodynamic biomarker for Aurora A-targeted drugs. Sensitive pharmacodynamic biomarkers are essential for determining appropriate drug doses in clinical trials, thus preventing unnecessary toxicity. In one embodiment a method of determining in vivo efficacy of an anti-Aurora A therapy is monitored by measuring the concentrations of LIMK2 during the course of the administration of the Aurora A inhibitor therapy. In one embodiment the method comprising the steps of measuring LIMK2 concentrations in a biological sample obtained from a patient receiving said anti-Aurora A therapy during the course of therapy. In one embodiment a baseline LIMK2 concentration is determined either based on population data, or for the specific patient prior to receiving the Aurora A inhibitor therapy. The LIMK2 concentrations are then determined at least once during the Aurora A inhibitor therapy. In one embodiment the detected LIMK2 concentrations are used to monitor the effectiveness of the Aurora A inhibitor therapy and the results are used to modify the therapy by increasing or decreasing dosages of the Aurora A inhibitor and/or alter the composition of the inhibitor being administered. In one embodiment the in vivo concentrations of LIMK2 can be used to select the most efficacious in vivo Aurora A inhibitor, or combination of Aurora A inhibitors. Over a dozen Aurora A inhibitors are in advanced clinical trials and are known to those skilled in the art. In a further embodiment the concentrations of PHLDA-1 in biological samples obtained from said patient can also be monitored during the administration of said anti-Aurora A therapy, wherein an increased concentration of PHLDA-1 is indicative of the effectiveness of the administered Aurora A inhibitor.

In accordance with one embodiment a method of determining in vivo efficacy of an anti-Aurora A therapy is provided wherein

(a) the concentration of LIMK2 proteins in a first biological sample is obtained from the patient;

(b) an anti-Aurora A therapeutic treatment is administered to the patient;

(c) a second biological sample is obtained from the patient after or during step (b);

(e) the concentration of LIMK2 proteins in the first and second biological sample is determined; and

(f) the concentration of LIMK2 proteins in the first and second biological samples is compared, wherein detection of LIMK2 levels in the second biological sample that are closer to those of healthy individuals, relative to the levels of LIMK2 detected in the first biological sample, is indicative of therapeutic efficacy. In one embodiment multiple samples are obtained from the patient over the course of the anti-Aurora A therapy, and LIMK2 concentrations are determined for each of the samples. In one embodiment the dosage of the anti-Aurora A therapy is modified based on the detected LIMK2 concentrations. In a further embodiment the concentration of PHLDA-1 in biological samples obtained from the patient are also monitored during the administration of said anti-Aurora A therapy, wherein detection of PHLDA-1 levels in biological samples obtained after initiating the anti-Aurora A therapy that are closer to those of healthy individuals, relative to the levels of PHLDA-1 detected in the first biological sample, is indicative of therapeutic efficacy.

In accordance with one embodiment compositions and methods are provided for diagnosing, determining therapeutic strategy, monitoring therapeutic efficacy and treating cancer, including breast and prostate cancer. In one embodiment the cancer to be treated is breast cancer. The method comprises the steps of measuring the relative concentration of PHLDA1 and/or LIMK2 proteins, or peptide fragments thereof, or nucleic acid sequences encoding the same in a biological sample obtained from a patient. The biological sample can be any body fluid such as blood or a solid tissue sample.

In accordance with one embodiment a method for diagnosing or determining a therapeutic strategy for treating cancer is provided based on Aurora A activity as indicated by PHLDA1 and/or LIMK2 in a biological sample obtained from a patient. In one embodiment the method comprises

(a) measuring the concentration of PHLDA1 and/or LIMK2 in a biological sample obtained from a patient, or peptide fragments of PHLDA1 and/or LIMK2, or nucleic acid sequences encoding the same in said biological sample;

(b) comparing the concentration of PHLDA1 and/or LIMK2, or peptide fragments or derivatives thereof, or nucleic acids encoding the same with a control; and

(c) determining whether the detected levels of PHLDA1 and/or LIMK2 in the biological sample relative to the control indicate a likelihood of cancer. In one embodiment the control represents the levels of PHLDA1 and/or LIMK2, or peptide fragments thereof, or nucleic acid sequences encoding the same, in a biological sample typical for healthy individuals. In one embodiment the cancer to be detected is breast cancer.

In accordance with one embodiment a method of monitoring the efficacy of a cancer therapy is provided wherein the method comprises the steps of measuring the concentration of PHLDA1 and/or LIMK2 proteins, or peptide fragments thereof, or nucleic acid sequences encoding the same in a biological sample obtained from a patient, administering a therapeutic composition or procedure to the patient and taking a second measurement of the concentration of PHLDA1 and/or LIMK2 proteins after the administration of the therapeutic composition or procedure. In one embodiment the therapeutic composition comprises an anti-Aurora A therapeutic (e.g., an inhibitor of Aurora A activity).

In accordance with one embodiment a method of inhibiting the proliferation of cells, and more particularly, the proliferation of neoplastic cells is provided. In one embodiment the neoplastic cells are cancer cells, including for example, breast, prostate, ovarian, colorectal and pancreatic cancer and in one specific embodiment the cancer cells are breast or prostate cancer cells. The method comprises contacting the cells with a composition comprising an LIMK2 inhibitor. In one embodiment the neoplastic cells are contacted with both a LIMK2 inhibitor and an Aurora A inhibitor. The LIMK2 inhibitor and Aurora A inhibitor can be administered simultaneously (e.g. as part of a single composition) or they can be administered sequentially.

In one embodiment a method of treating cancer is provided wherein the method comprises the step of increasing the expression, stability or activity of PHLDA1 in target cancer cells. In one embodiment a method of treating cancer is provided wherein the method comprises the step of decreasing the expression, stability or activity of LIMK2 in target cancer cells. In accordance with one embodiment the cancer to be treated is selected from the group consisting of colorectal, pancreatic, breast and prostate cancer. In one embodiment the cancer to be treated is breast cancer.

In accordance with one embodiment the activity of PHLDA1 or LIMK2 is modified in target cancer cells using recombinant techniques. For example, in one embodiment the expression of PHLDA1 can be enhanced by the administration of small molecules such as estrogen. In another embodiment the expression of PHLDA1 can be enhanced by introducing additional copies of the PHLDA1 gene under the control of strong and/or inducible promoters. Suitable in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. In one embodiment the constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Alternatively, the activity of PHLDA1 and/or LIMK2 can be modulated by chemical means, using compounds that are known to modulate the activity of PHLDA1 and/or LIMK2. In one embodiment compounds are administered that inhibit or interfere with the activity of LIMK2. Such agents include for example the use of small interfering RNA (siRNA) or antisense nucleic acid sequences. Use of small interfering RNA (siRNA) is a two-step process. The first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 by duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to form the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Synthesis of RNAi molecules suitable for use with the present invention can be effected as follows. First, an LIMK2 mRNA sequence, for example, is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (for details see the Ambion Inc. web site, item “techlib/tn/91/912”).

Second, potential target sites are compared to an appropriate genomic database (e.g., human, mouse, rat etc.) using any sequence alignment software, such as the BLAST software available from the NCBI server (see “BLAST” at the NCBI.gov website). Putative target sites which exhibit significant homology to other coding sequences are filtered out. Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation.

For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Another agent capable of downregulating LIMK2 or an effector thereof is a DNAzyme molecule capable of specifically cleaving an mRNA transcript or DNA sequence of interest. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al, the disclosure of which is incorporated herein by reference. DNAzymes of similar design directed against the human Urokinase receptor were recently observed to inhibit Urokinase receptor expression, and successfully inhibit colon cancer cell metastasis in vivo (Itoh et al, 2002, Abstract 409, Ann Meeting Am Soc Gen Ther, available at the American Society for Gene Therapy website). In another application, DNAzymes complementary to bcr-ab1 oncogenes were successful in inhibiting the oncogenes expression in leukemia cells, and lessening relapse rates in autologous bone marrow transplant in cases of CML and ALL.

Reducing LIMK2 activity or an effector thereof can also be effected by using an antisense polynucleotide capable of specifically hybridizing with an mRNA transcript encoding the proteins of interest. Design of antisense molecules which can be used to efficiently downregulate a gene product of interest must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof A number of delivery strategies are known to those skilled in the art which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bc1-2 had entered clinical trials and had been shown to be tolerated by patients [Gerwitz Curr Opin Mol Ther 1:297-306 (1999)]. More recently, antisense-mediated suppression of human heparanase gene expression has been reported to inhibit pleural dissemination of human cancer cells in a mouse model [Uno et al., Cancer Res 61:7855-60 (2001)].

Another agent capable of reducing the expression of LIMK2 activity or an effector thereof is a ribozyme molecule capable of specifically cleaving an mRNA transcript encoding this gene product. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials.

In one embodiment LIMK2 inhibition is achieved using standard techniques know to those skilled in the art including the use of small molecule inhibitors (see for example Harrison et al. (2009) Novel class of LIM-kinase 2 inhibitors for the treatment of ocular hypertension and associated glaucoma. J Med. Chem. 2009 Nov. 12; 52(21):6515-8, the disclosure of which is incorporated herein).

### Example 1

Identification of PHLDA1 as a Negative Regulator and Effector of Aurora A Kinase.

Materials and Methods:

Antibodies for Aurora A (H-130), actin (C-2), a-tubulin (B-7) and PHLDA 1 (L-19), phospho-histone H3 were purchased from Santa Cruz Biotech.

Expression Plasmids and Constructs

Aurora A-as1 (L201G) and Aurora A-as7 (LI85V, L201G) were generated using overlapping PCR and cloned using BamHI and NotI sites in a VIP3 puro retroviral mammalian vector and a baculoviral vector (Bac to Bac, Invitrogen). PHLDA I was cloned in TAT-HA and VIP3 vectors at BamHI and Xho 1 sites. TPX2 (a gift from Dirk Gorlich) was cloned into Fastbac vector at BamHI and Kpn 1 sites.

Expression and Purification of TPX2, Wt Aurora A, Aurora A Mutants and PHLDA1

For substrate labeling experiments, Aurora A (AA), AA-as1, AA-as7 and TPX2 were prepared from Sf9 insect cells using the baculovirus Bac-to-Bac expression system (Invitrogen) according to the manufacturer's instructions. Protein concentration was determined using Bradford assay, and the protein purity was assessed using 6-His antibody. PHLDA1 was expressed in E. coli and purified as describe before (Sun, et al, (2008a) Mol. Biol. Cell 19, 3052-3069).

2D Gel Electrophoresis and MS Spectrometry

To prepare a labeled sample for 2D gel electrophoresis, kinase reactions with whole cell lysate or fractionated lysate were carried out as published before (Sun, et al, (2008a) Mol. Biol. Cell 19, 3052-3069). Gel spots were manually excised and automatically processed for peptide mapping experiments using a Micromass MassPREP Station in conjunction with manufacturer specified protocols.

Synthesis of [γ-³²P] N⁶(Phenethyl) ATP and l-NM-PP1

[γ-³²P] N⁶(Phenethyl) ATP and 1-NM-PP1 were synthesized as described before (Shah and Shokat (2002) Chem. Biol. 9, 35-47).

Transfection and Retroviral Infection

Aurora A, PHLDA1 and TPX2 plasmids were transiently transfected into Phoenix cells. The retroviruses were harvested and used to infect MDA-MB-231 cells as reported previously (Shah and Shokat (2002) Chem. Biol. 9, 35-47).

In Vitro Kinase Assays:

For in vitro labeling, Aurora A/TPX2 complex (on beads) was pre-incubated with 10 μM cold ATP for 10 min to activate the kinase. The beads were washed twice with kinase buffer, and then subjected to kinase assay with 2-5 μg of recombinant protein (such as PHLDA) and 1 μCi of [γ-³²P] ATP. Reactions were terminated by adding SDS sample buffer, separated by SDS-PAGE gel, transferred to PVDF membrane and exposed to Biomax MS film. Aurora A kinase assays were conducted using 1 μCi of [γ-³²P] ATP and 3 μg of Aurora A substrate peptide in a final volume of 30 μl at 30° C. as reported before (Sun, et al, (2008a) Mol. Biol. Cell 19, 3052-3069).

IC₅₀ values were determined by fitting the data to a sigmoidal dose response curve using GraphPad Prism 4.0 software. Km and Vmax values were derived from the assay described above using various concentrations of AA peptide substrate and ATP. Km and Vmax values were determined using GraphPad Prism 4.0 software.

Aurora A and PHLDA1 shRNA

Aurora A human short hairpin RNA (shRNA) sequences were designed as follows: (1) forward oligo 5′-CCGGGCACCACTTGGAACAGTTTATCTCGAGAT AAACTGTTCCAAGTGGTGCTTTTTG -3′ (SEQ ID NO: 11) and reverse oligo 5′-AATTCAAAAAGCACCACTTGGAACAGTTTATCTCGAGATAAACTGTTCCAAGTGGT GC-3′ (SEQ ID NO: 12); (2) 5′ CCGGGCCAATGCTCAGAGAAGTACTCT CGAGAGTACTTCTCTGAGCATTGGCTTTTTG-3′ (SEQ ID NO: 13) and reverse oligo 5′-AATTCAAAAAGCCAATGCTCAGAGAAGTACTCTCGAGAGTACTTC TCTGAGCATTGGC-3′ (SEQ ID NO: 14). For PHLDA I, following sequences were designed: (1) forward oligo 5′-CCGGGATGGTGCAGTACAAGAATCTCGAGATTCT TGTACTGCACCATCTTTTTG-3′ (SEQ ID NO: 15) and reverse oligo 5′-AATTCAAAAAGATGGTGCAGTACAAGAATCTCGAGATTCTTGTACTGCACCATC-3′ (SEQ ID NO: 16). (2) forward oligo 5′-CCGGTCCGCATCCACA TCCACATCTCGAGATGTGGATGTGGATGCGGATTTTTG-3′ (SEQ ID NO: 17) and reverse oligo 5′-AATTCAAAAATCCGCATCCACATCCACATCTCGAGATGTGGATGT GGATGCGGA-3′ (SEQ ID NO: 18). The sense and antisense strands were annealed at 95° C. for 4 minutes to make at 20 μM concentration. This was followed by cooling to room temperature and subsequently cloned into pLKO.1 TRC vector (Moffat et al., 2006). pLKO.1 TRC vector was a gift from David Root. Control shRNA (scrambled shRNA) and AA shRNA Aurora A were transfected to MDA-MB-231 cells using Lipofectamine following manufacturer instructions. After 30 h, transfected cells were harvested and analyzed for AA and PHLDA1 expression. Alternatively, AA shRNA and PHLDA1 lentiviruses were generated and used for infecting MDA-MB-231 cells.

Chemotaxis Assay

MDA, AA-MDA, PHLDA1-MDA and PHLDA1 and AA overexpressing MDA cells were serum starved in serum-free RPMI for 12 h and isolated by limited trypsin digestion. Cell migration was determined as we reported before (Shah and Vincent (2005) Mol. Biol. Cell 16, 5418-5432). The assays were performed in triplicate, four independent times. To allow for comparison between multiple assays, the data were normalized, and expressed as a percentage of the number of cells present on the membrane.

Soft Agar Colony Formation:

Briefly equal volumes of nobel agar (1%, DNA grade) and 2×RPMI 1640 (with 20% FBS) were mixed at 40° C. to make 0.5% agar in six-well tissue culture plates (Corning) as a base agar. Cells (0.1 ml of 2.0×10⁵/ml) were suspended in 3 mL of 2×RPMI 1640 (with 20% FBS) and 3 ml of 0.7% agar. 1.5 ml of this suspension was added to each well (as 0.35% top agar) with final concentration of 5,000 cells per well. Top agar was covered with 500 μl of culture media. Plates were incubated at 37° C. for 3 to 4 weeks. Fresh media was added every three days.

For 1-NM-PPI experiments, fresh media containing 1-NM-PP1 (100 nM) or DMSO were added to the cells every 3 days. Colony formation was observed by light phase-contrast microscope and visually after staining with 0.5 ml of 0.01% crystal violet in PBS for 45 min at room temperature. Experiments were repeated in quadruplicate, two independent times to ensure the reproducibility of the results.

Cell Synchronization

MDA or PHLDA1-overexpressing MDA (PHLDA1-MDA) cells were treated with 2.5 mM thymidine for 16 h, released for 8 h, and then treated with thymidine for an additional 16 h. After two washes with phosphate-buffered saline (PBS), cells were cultured for different times as indicated in the experiment and harvested.

Immunoflorescence

MDA and PHLDA 1-MDA cells were plated on poly-L-Lysine-coated coverslips at a density of 50,000 cells per well in 24-well plates. Cells were arrested at G1/S using double thymidine block, followed by release for different time periods. Cells were immunostained using α-tubulin, phospho-histone H3 (S10), Aurora A or PHLDA1 antibodies, followed by FITC-labeled goat anti-rabbit or Texas red-labeled goat anti-mouse secondary antibodies. After washing with PBS, coverslips were mounted on microscope slides with Mowiol mounting medium. Images were taken using a Fluoview laser scanning confocal microscope (Olympus, Melville, N.Y.). The percentages of cells shown were counted in at least 100 cells from ten random frames in duplicate.

Peptide Synthesis

Aurora A substrate peptide, ALRRASLGAA (SEQ ID NO: 19), was synthesized using solid-phase peptide synthesis using a standard Fmoc peptide synthesis protocol and WANG resin.

MTT Assay

Cells were seeded in 96-well plate at 1,500 cells per 100 μL per well and cultured for 24, 48, and 72 h. At the end of incubation, MTT assay was conducted as published previously (Sun, et al. (2009) Mol. Biol. Cell 20, 4611-4619). Experiments were repeated three times in quadruplicate wells to ensure the reproducibility of results.

Molecular Modeling:

Using PDB number 1MQ4, the docking of phenethyl ATP was carried out using MacroModel. Amino acids L194 and L21 0 (human Aurora A numbering) were mutated to Valine and Glycine residues, respectively. The resulting structure was then energy minimized to yield a unique favorable conformation visualized by pymol.

Immunohistochemical Studies:

In order to study the significance of PHLDA1 in human tissues, we analyzed a breast cancer tissue microarray (TMA) for protein expression using immunohistochemistry (IHC). The TMA was created at Indiana University, Department of Pathology. It consists of 1 nun tissue cores from 114 breast cancer patients treated at this institution. Data regarding age, tumor type (IDC vs. ILC/others), grade (I vs. II vs. III), tumor size (2 cm vs. >2 cm), nodal status (No vs. Yes), ER (Neg vs. Pos), PR (Neg vs. Pos) and HER2 (Neg vs. Pos) status were available for this cohort. Expression of PHLDA1 was analyzed using mouse antihuman PHLDA1 monoclonal antibody (Santa Cruz Biotech, Santa Cruz, Calif.) by IHC. After de-waxing and hydration, 4 mm sections from formalin-fixed paraffin embedded tissue were treated with target retrieval (Dako, pH 8.0), in a pressure cooker. Endogenous peroxidase activity was blocked by hydrogen peroxide for 10 min. The slides were then incubated with mouse monoclonal PHLDA 1 antibody (1:50; Santa Cruz Biotech) for 1 h at room temperature. The sections were incubated with donkey antigoat horseradish peroxidase polymer conjugate (Jackson Labs, West Grove, Pa.) according to the manufacturer's instructions. The stain was developed using diaminobenzidine (DAB) plus (Dako, Glostrup, Denmark) and hematoxylin QS (Vector Laboratories, Burlingame, Calif., USA) counterstain. To verify the specificity of staining, nonimmune goat serum and PBS-negative controls were used. Expression of PHLDA1 was evaluated for intensity of staining and scored as 0 (no expression), 1 (weak expression), 2 (moderate expression) and 3 (strong expression) by a single board certified pathologist (SB).

Statistical Analysis:

All statistical analyses were performed using SPSS v. 17.0. Expression of PHLDA1 protein was correlated with clinico-pathological variables as mentioned above using Chi-square test, Fishers test or Student's t test as appropriate. Bar graphs results are plotted as the average±SEM. Significant results are displayed as follows: *p>0.05, **p>0.01, ***p>0.001.

Results:

Cloning and Characterization of Analog-Sensitive Aurora A (AA-ad) Kinase:

An analog sensitive mutation was created in the Aurora A (AA) active site by replacing the gatekeeper residue L201 with a glycine residue (AA-as1 kinase). To identify the most optimal orthogonal phospho-donor for the engineered kinase, several [γ-³²P] ATP analogs were synthesized and screened using wt Aurora A and engineered AA-as1 kinase. AA-as1 kinase assay was conducted in the presence of 6-His tagged-TPX2, which is an Aurora A activator. Although AA-as 1 kinase displayed high kinase activity, it poorly accepted any of the ATP analogs. These results were surprising, since mutation of this single gatekeeper residue to G or A has been shown to confer analog-sensitivity in over 30 kinases.

Engineering A Novel mutation in Aurora A Kinase: Generation of A New Analog-Sensitive AA-as7 kinase

For the rational design of a mutant Aurora A kinase that is sensitive to orthogonal ATP analogs, modeling studies were conducted using the published crystal structure of human Aurora A bound to ADP (at 2.5 Å resolution). The goal was to introduce subtle changes in the ATP binding pocket already possessing the gatekeeper mutation (L201G, for murine Aurora A). The residues considered for mutagenesis had to be located near the N-6 position of the adenine ring, since gatekeeper residue is in close contact with N-6 position. Furthermore, ATP analogs possessing bulky groups are modified at N-6. These criteria suggested that an additional mutation at LI85 to a smaller residue would allow the engineered kinase to utilize orthogonal ATP analogs. LI85 is within 4 Å and gatekeeper L201 within 5 Å of the N-6 amino group. Sequence alignment of Aurora A with other kinases including v-Src revealed that most kinases engineered previously to generate analog-sensitive alleles possess a Val residue at this position. Interestingly, Ip11 kinase, a yeast homolog of aurora kinases, possesses Thr residue at this position, which is isosteric to Val. We postulated that the combined mutation of these two residues (LI85V, L201G) would produce a mutant possessing a hydrophobic cavity that would enable it to accept orthogonal ATP analogs and inhibitors.

Double mutant of Aurora A (LI85V, L201G) was generated and expressed in insect cells. Since previous studies have identified several mutations for generating analog-sensitive kinases (as-1, as-2, as-3, as-4, as-5 and as-6), we named this new mutant as “Aurora A-as-7” (AA-as7).

Catalytic Efficiency of Wild-Type and AA-as7 With ATP and N′6-(Phenethyl) ATP:

Kinase assays were conducted in the presence of TPX2 and [γ-³²P]-labeled N6-modified ATP analogs, which revealed N-6-Phenethyl ATP as the most optimal orthogonal phosphodonor for AA-as7 kinase. The catalytic efficiency of the AA-as7 kinase with N⁶-Phenethyl ATP (A*TP) (Kcat/Km=1.16×10³ min⁻¹ M⁻¹) was comparable to the efficiency of the mutant with ATP (Kcat/Km=1.84×10² min⁻¹ M⁻¹). More importantly, catalytic efficiency of AA-as7 was similar to the efficiency of wild-type AA with ATP (Kcat/Km=1.16×10² min⁻¹ M⁻¹) (Table 1).

TABLE 1
Kinetic data of wild-type Aurora A and AA-as7 with ATP
and N⁶-phenethyl ATP

| | |
|---|---|
| AA-as7 | Wild-type |

| | N⁶- | | Aurora A |
|---|---|---|---|
| | phenethyl ATP | ATP | ATP |
| Vₘₐₓ | 3.02 × 10⁶ | 7.31 × 10⁵ | 4.24 × 10⁵ |
| Kₘ | 11.58 | 17.70 | 16.21 |
| (μM) | | | |
| K_{cat} | 1.34 × 10⁴ | 3.25 × 10³ | 1.88 × 10³ |
| (minute⁻¹) | | | |
| Eₜₒₜₐₗ | 225 | 225 | 225 |
| (μg) | | | |
| K_{cat}/Kₘ | 1.16 × 10³ | 1.84 × 10² | 1.16 × 10² |

Screening of Orthogonal Inhibitors to Identity the Optimal Orthogonal Inhibitor For -as1 and -as7 Kinases:

A set of orthogonal inhibitors were synthesized and screened against the engineered kinases AA-as1 and AA-as7 to identify the most potent inhibitor. AA-as1 kinase was poorly inhibited, similar to the results obtained using ATP analogs, suggesting that the gatekeeper mutation alone is not enough to confer PP1-derived inhibitor-sensitivity to AA kinase. However, AA-as7 kinase was strongly inhibited. 1-NM-PP1 was identified as the most potent and specific inhibitor of the AA-as7 kinase (IC₅₀=1.7 nM).

Characterization of A New Analog-Sensitive Mutant of Aurora A (AA-as7):

Previous studies have shown that Aurora A overexpression in NIH3T3 cells causes cellular transformation, which depends on its kinase activity. Therefore, wild-type Aurora A and AA-as7 were overexpressed in NIH3T3 cells at similar levels to evaluate their relative transformation efficiency. Both wild-type and mutant Aurora A expressing NIH3T3 cells showed similar transformation potential in a soft agar assay, suggesting that the double mutations in AA-as7 kinase are functionally silent.

1-NM-PP1 Inhibits Colony Formation in AA-as7-NIH3T3 cells, not in Wt AA-NIH3T3 Cells:

Our in vitro data showed that 1-NM-PP1 is highly potent and selective for AA-as7 kinase (Table 1). To confirm this specificity in cells, AA-as7-NIH3T3 cells were treated with 0.5 μM 1-NM-PP1, which completely inhibited colony formation. Under identical conditions, wild-type Aurora A-expressing cells demonstrated robust colony formation in soft agar assay. This result confirmed that 1-NM-PP1 is highly orthogonal and only inhibits AA-as7 kinase.

Aurora B Also Requires Double Mutations For Generating Analog-Specific Mutant:

We next generated Aurora B-as1 kinase by mutating gatekeeper L154 to G. Similar to AA-as1 mutant, AB-as 1 mutant also poorly accepted ATP analogs and PP I-derived inhibitors. Since Aurora B also possesses a Leu residue at 138 position (equivalent to LI85 of AA), it was mutated to Val and the double mutant (L138V, L154G) analyzed. Aurora B-as7 (L138V, L154G-AB) kinase showed highest catalytic efficiency using N-6-Phenethyl ATP, which was comparable to wild type with ATP (Table 2).

TABLE 2
Kinetic data of wild-type Aurora B and AB-as7 with ATP
and N⁶-phenethyl ATP

| | |
|---|---|
| AB-as7 | Wild-type |

| | N⁶- | | Aurora B |
|---|---|---|---|
| | phenethyl ATP | ATP | ATP |
| Vₘₐₓ | 7.44 × 10⁴ | 3.30 × 10⁴ | 5.12 × 10⁴ |
| Kₘ | 11.4 | 30.4 | 23 |
| (μM) | | | |
| K_{cat} | 372 | 165 | 256 |
| (minute⁻¹) | | | |
| Eₜₒₜₐₗ | 200 | 200 | 200 |
| (μg) | | | |
| K_{cat}/Kₘ | 32.63 | 5.42 | 11.13 |

These results suggest that residue LI85 is an important determinant of analog-sensitivity in kinases. A small decrease in the side chain from Leu to Val is essential (in addition to the gatekeeper mutation) for generating a pocket capable of accepting orthogonal ATP analogs and inhibitors. Although, a majority of protein kinases possess Val at this position, there are a few which possess either Ile or Leu. Thus, the next generation mutant strategy (as7 kinases) should be beneficial in conferring analog-sensitivity to this class of kinases.

Global Screen Using MDA-MB-231 (MDA) Cell Lysate and AA-as7 Kinase Revealed PHLDA1 As a Novel Aurora A Substrate:

An in vitro kinase reaction was performed using unsynchronized MDA cell lysate, [[γ-³²P] Phenethyl-ATP and Aurora A-as7/TPX2 to identify novel Aurora A substrates. Addition of Phenethyl-ATP to the cell lysate alone showed no signal as expected. Phenethyl ATP is specific for the engineered kinase and is not accepted by wt kinases present in the cell lysate. In contrast, when AA-as7 and TPX2 were added to the cell lysate along with Phenethyl ATP, it phosphorylated several proteins. These proteins are the direct targets of Aurora A. To isolate these targets, proteins were separated using 2D gel electrophoresis, isolated and visualized by autoradiography. Radiolabeled proteins were excised from gel and subsequent to trypsin digestion the peptide cleavage products were identified by tandem mass spectrometry. Several Aurora A substrates were identified including PHLDA1, p53 and vimentin. As disclosed herein Pleckstrin homology-like domain, family A, member 1 (PHLDA1 aka TDAG51) was further investigated as a potential target of Aurora A. PHLDA1 gene has been shown to be downregulated in majority of breast cancer tumors and is a strong predictor of poor prognosis for breast cancer patients.

PHLDA1 is Directly Phosphorylated by Aurora A:

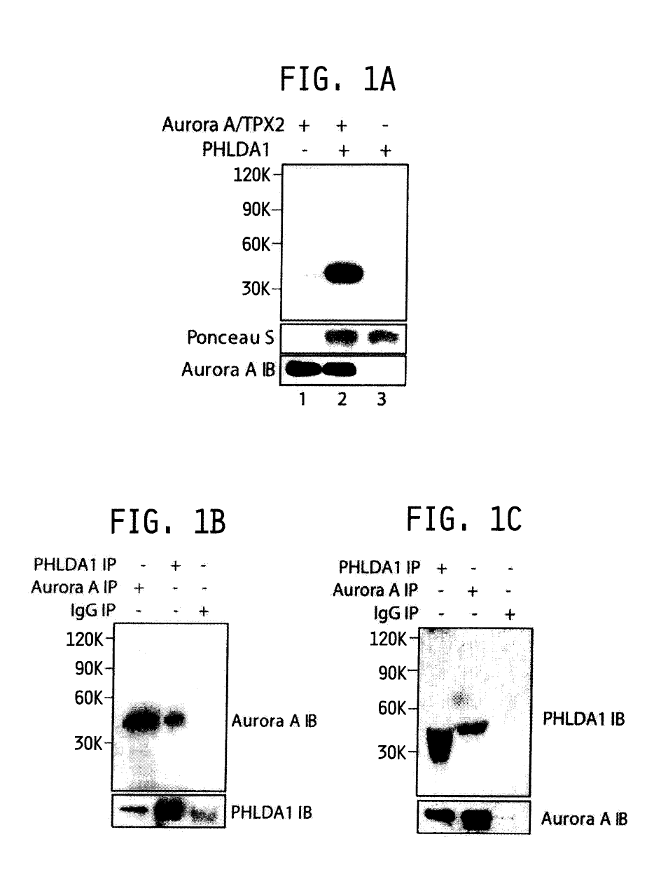

Since proteomics screen can often lead to false positives, we investigated Aurora A-mediated phosphorylation of PHLDA1 using an in vitro kinase assay. PHLDA1 was generated as 6-His fusion protein and subjected to in vitro kinase assay with Aurora A/TPX2. Aurora A directly phosphorylated PHLDA1 (FIG. 1A, lane 2).

PHLDA1 and Aurora A Associate in MDA-MB-231 (MDA) cells: Kinase substrate specificity in vivo is maintained by cellular localization and protein-protein interactions. As a result, cellular fractionation and in vitro kinase assays may lead to artifacts. Therefore, PHLDA1 and Aurora A association was analyzed within the cells. PHLDA I immune complexes were isolated and Aurora A binding determined. Aurora A was co-immunoprecipitated with PHLDA 1 (FIG. 1B). Similar results were obtained when Aurora A immune complex was isolated and PHLDA1 binding analyzed (FIG. 1C). These findings show that Aurora A and PHLDA1 associate with each other in MDA cells. PHLDA1 and Aurora A Co-localize in MDA cells: Aurora A and PHLDA1 localization was examined in unsynchronized MDA cells, which revealed cytoplasmic localization for both.

Aurora A Negatively Regulates PHLDA1 Levels Using its Kinase Activity:

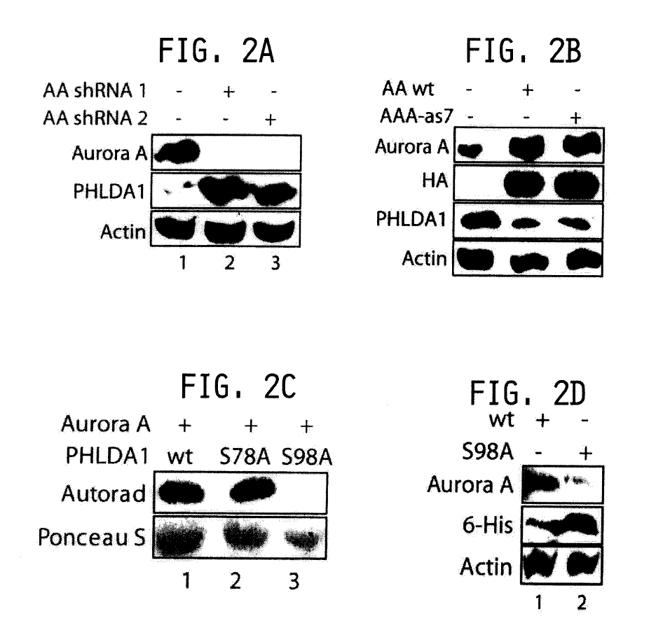

Aurora A-mediated phosphorylation often promotes degradation (ex. p53, NDELI) or stabilization of its substrates (AlP, HURP, ASAP1. Therefore, we investigated whether Aurora A exerts a control over PHLDA1 level. Two different Aurora A shRNAs were generated and used to ablate it in MDA cells. Aurora A depletion increased PHLDA1 level significantly in both cases (FIG. 2A, middle panel), suggesting Aurora A degrades PHLDA1. To confirm this finding, wild-type (AA-MDA) and AA-as7-overexpressing stable MDA cells (AA-as7-MDA) were generated and PHLDA1 expression determined. Aurora A overexpression significantly decreased PHLDA1 levels (FIG. 2B). Together these results show that Aurora A negatively regulates PHLDA I level.

Aurora A can regulate its substrates using either its kinase activity or scaffolding function. Therefore, to dissect the mechanism further, AA-MDA and AA-as7-MDA were treated with 1-NM-PP1 for 12 h and PHLDA1 expression analyzed. 1-NM-PP1 treatment increased PHLDA1 level only in AA-as7-MDA cells, but not in wt Aurora A-MDA cells. Since 1-NM-PP1 selectively inhibits AA-as7 kinase activity, and not wild-type Aurora A (Table I), this finding suggests that Aurora A-mediated down regulation of PHLDA I is due to its kinase function, and not due to its scaffolding interactions. Interestingly, we also observed decreased Aurora A levels in 1-NM-PP 1-treated cells, suggesting that Aurora A inhibition may have a negative impact on its protein levels as well.

Aurora A Negatively Regulates PHLDA1 Levels by Directly Phosphorylating Ser98:

Aurora A preferentially phosphorylates R/K/N-R-X-S/T-B, where B denotes any hydrophobic residue except Pro. This preference revealed Ser78 and Ser98 as putative Aurora A phosphorylation sites on PHLDA1. Ser78A and Ser98A PHLDA1 alleles were generated and their phosphorylation analyzed using Aurora A/TPX2. Aurora A was found to predominantly phosphorylate PHLDA1 at Ser98 (FIG. 2C).

To elucidate the functional significance of this phosphorylation, both wt 6-His-PHLDA1 and 6-His-Ser98A-PHLDA1 were transfected in Aurora A-MDA cells, and their levels analyzed after 24 h using 6-His antibody. While Ser98A PHLDA1 showed robust expression in Aurora A-MDA cells, wt PHLDA1 was degraded significantly, presumably due to Aurora A-mediated phosphorylation at Ser98 (FIG. 2D). These findings suggest that Aurora A downregulates PHLDA1 by phosphorylating Ser98. Since Aurora A is an oncogene and PHLDA1 promotes apoptosis, PHLDA1 downregulation may be one of the mechanisms by which Aurora A promotes breast tumorigenesis.

PHLDA1 Negatively Regulates Aurora A Levels:

A few Aurora A substrates are known to regulate Aurora A activity/expression in a feedback mechanism. Aurora A phosphorylates FAF1, which in turn degrades Aurora A. Similarly, PPI inhibits Aurora A kinase activity upon phosphorylation by Aurora A. Since we observed lower levels of Aurora A in Ser98A-PHLDA1-MDA cells as compared to wt-PHLDA1-MDA cells, it suggested that PHLDA1 may also negatively regulate Aurora A. Furthermore, lower Aurora A levels were observed when Aurora A was inhibited in AA-as7-MDA cells using 1-NM-PP 1, suggesting that increase in PHLDA I levels may in turn inhibit Aurora A in a negative feedback loop.

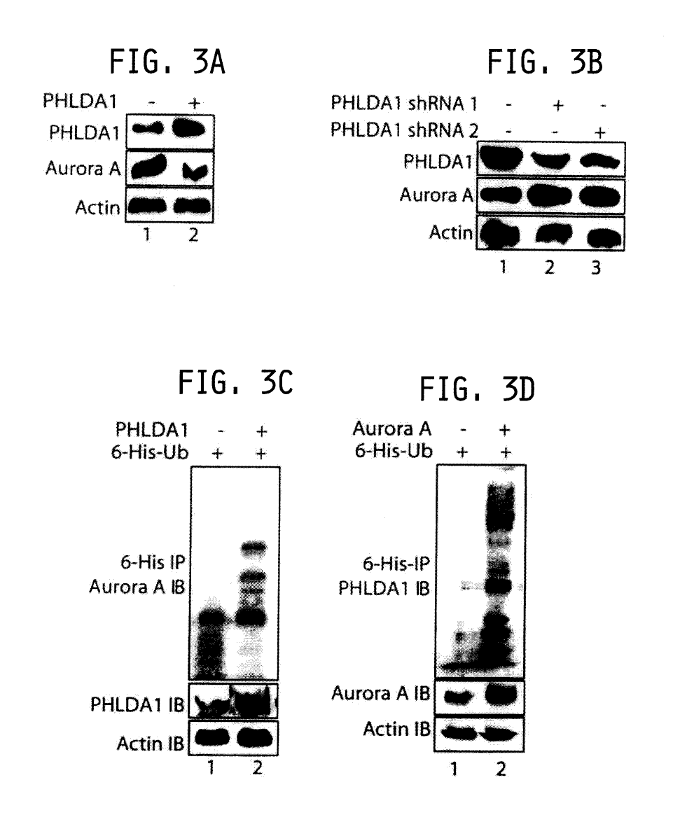

PHLDA1 was stably expressed in MDA (PHLDA1-MDA) cells and Aurora A levels analyzed. PHLDA1 overexpression decreased Aurora A level (FIG. 3A). Next two different PHLDA1 shRNAs were generated and used to reduce PHLDA1 levels in cells, which increased Aurora A level significantly (FIG. 3B). These findings show that PHLDA1 negatively regulates Aurora A levels, presumably by recruiting degradation machinery.

To probe whether Aurora A degradation by PHLDA1 is mediated by ubiquitination, 6-His-ubiquitin was transfected in MDA and PHLDA1-MDA cells and Aurora A ubiquitination analyzed using 6-His antibody. PHLDA1 overexpression indeed increased ubiquitinated Aurora A. In parallel, PHLDA1 degradation was analyzed in MDA and AA-MDA cells, which also showed increased ubiquitination of PHLDA1 upon Aurora A overexpression. These results demonstrate that Aurora A and PHLDA1 negatively regulate each other by promoting each other'subiquitination (FIG. 3D).

Ubiquitination levels of wt PHLDA 1 and S98A PHLDA 1 in AA-MDA cells were then examined, which revealed increased ubiquitination of wt, but not phosphorylation-resistant S98A PHLDA1 allele. These findings further confirm that Aurora A degrades PHLDA1 by direct phosphorylation at Ser98.

PHLDA1 is not a Mitotic Target of Aurora A

PHLDA1's role in mitosis has not been analyzed. Since Aurora A is predominantly expressed during mitosis in normal cells, we investigated whether PHLDA 1 has a role in mitosis. We initially analyzed the protein levels of PHLDA 1 in synchronized MDA cells. MDA cells arrested at G1/S using double thymidine block were released for varying periods, and PHLDA1 and Aurora A levels analyzed. Interestingly, PHLDA1 was expressed almost uniformly expressed throughout the cell cycle, whereas Aurora A expression markedly increased during mitosis, suggesting that PHLDA1 expression is not cell cycle regulated.

Nevertheless, to examine a potential role of PHLDA1 in mitosis, FACS analysis was conducted using unsynchronized MDA and stable PHLDA1-MDA cells. Both cell-types showed similar distribution of cells in different cell cycle phases and no aneuploidy, which further supported the notion that PHLDA1 overexpression does not affect cell cycle.

Since FACS analysis may not differentiate subtle changes in cell cycle, histone H3 (Ses10) phosphorylation (a mitotic marker) was next analyzed in synchronized MDA and PHLDA1-MDA cells. Both MDA and PHLDA1-MDA cells showed similar percentage of phosphohistone H3-positive cells at different time upon release from thymidine block, further suggesting that PHLDA1 is not a mitosis-regulated target of Aurora A. Importantly, in PHLDA1-MDA cells, PHLDA1 overexpression reduced Aurora A levels, but did not ablate it fully (see FIG. 3A), suggesting that reduced Aurora A levels are sufficient to carry normal mitotic functions in MDA cells.

Since Aurora A plays a vital role in mitotic spindle formation, we further examined the consequences of PHLDA1 overexpression in mitotic spindle assembly. MDA and PHLDA1-MDA cells arrested at G1/S were released for varying periods, and mitotic spindle analyzed. PHLDA1 overexpression did not affect mitotic spindle assembly in majority (˜95%) of the cells. Since PHLDA1-MDA cells are pooled population, variable PHLDA1 and Aurora A levels (due to the negative feedback loop) are expected in these cells. Therefore, it appears that in 5% of cells showing defective mitotic spindle, it is presumably due to very low levels of Aurora A. Together these results show that PHLDA1 is not directly involved in mitosis, however, it may indirectly affect it by negatively regulating Aurora A levels.

PHLDA1 is a Negative Regulator of Aurora A-Mediated Breast Oncogenesis:

PHLDA1 reportedly acts both as an apoptotic and as an anti-apoptotic agent. In NIH3T3 cells expressing IGF 1 receptors, PHLDA1 expression is essential for rescuing cells from serum starvation-induced apoptosis. On the contrary, in many other cell lines including T cells, neuronal, endothelial, melanoma, and cervical carcinoma, PHLDA1 reduces proliferation and induces cell death. In ras-transformed HME16C cells, reducing PHLDA1 protein level enhances cell growth under anchorage-independent, but not attached conditions. Furthermore, loss of PHLDA1 also favors apoptosis in these cells, although not significantly. The mechanism by which PHLDA1 may affect breast malignancy has not been analyzed.

To dissect the role of PHLDA1 in breast cancer cells, wild-type PHLDA1 and (S98A) PHLDA1 were overexpressed in MDA-MB-231 cells and cell proliferation was determined; both reduced cell growth, but (S98A) PHLDA1 inhibited it more. These results differ from the previous findings in breast epithelial HME16C cells, which revealed no change in cell proliferation rate upon PHLDA1 downregulation under attached conditions.

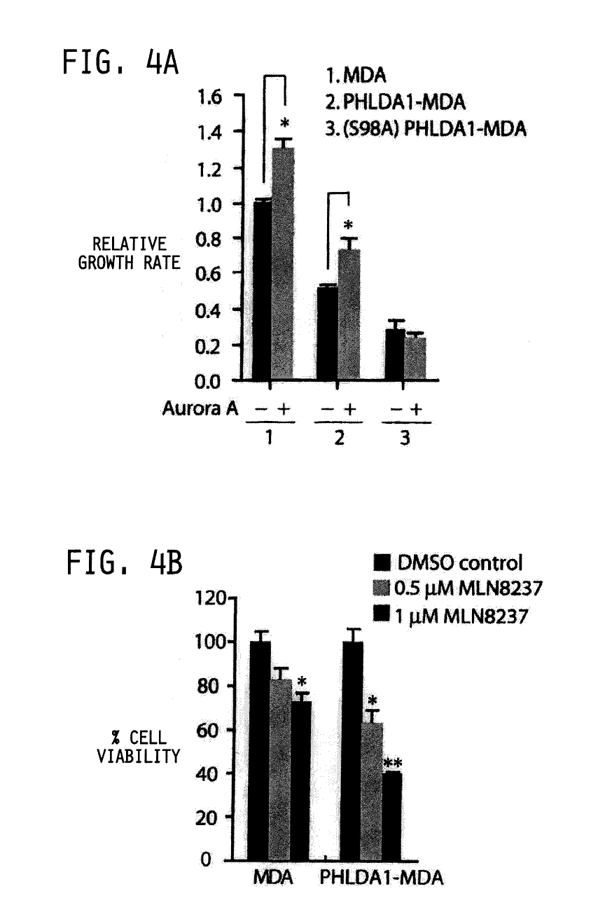

The reverse experiment was conducted by transiently transfecting Aurora A in control MDA-MB-231 cells or in MDA-MB-231 cells overexpressing wild-type PHLDA1 or (S98A) PHLDA1 and cell proliferation was measured (FIG. 4A). Although Aurora A expression increased cell proliferation in both control and PHLDA1-MDA cells, it did not affect the proliferation rate of (S98A) PHLDA1-MDA cells (FIG. 4A). This result was expected, because (S98A) PHLDA1 is resistant to Aurora-A-mediated degradation; instead, it efficiently degrades Aurora A. The effect of PHLDA1 was further evaluated in control and Aurora A-MDA cells under anchorage-independent conditions, which revealed dramatic loss in colony-forming ability upon PHLDA1 overexpression in both cell types. Thus, PHLDA1 upregulation inhibits cell proliferation under attached, as well as anchorage-independent, conditions in breast cancer cells. More importantly, the striking increase in the colony forming ability of MDA-MB-231 cells upon Aurora A overexpression was almost completely lost upon PHLDA1 overexpression suggesting that downregulation of PHLDA1 is one of the key mechanisms by which Aurora A promotes tumorigenesis.

PHLDA1 is a Negative Regulator of Chemotaxis:

PHLDA1's role in cell motility has not been investigated. To examine a potential contribution of PHLDA1 in Aurora A-mediated cell invasion, cell motility was measured in serum-starved AA-MDA and PHLDA1-AA-MDA cells. While AA-MDA cells were highly motile, PHLDA1 overexpression dramatically reduced cell motility in PHLDA1-AA-MDA cells. This result was further confirmed using a wound healing assay, which also revealed PHLDA 1 as a strong inhibitor of chemotaxis. As chemotaxis is important in cancer metastasis, these results show that PHLDA 1 is a key tumor suppressor in breast malignancy.

PHLDA1 Up Regulation and Aurora A Inhibition Act Synergistically in Promoting Cell Death:

PHLDA1's negative role in Aurora A-mediated oncogenic pathways suggested that PHLDA1 upregulation may work synergistically with Aurora A inhibition in promoting cell death. Aurora A was inhibited using VX-680 in MDA and PHLDA1-MDA cells. While ˜20% loss in cell viability was observed in MDA cells, >60% was observed in PHLDA1-MDA cells (see FIG. 4B). These findings suggest that PHLDA1 upregulation may be an alternative approach to modulate Aurora A-mediated breast oncogenesis.

PHLDA1 protein expression correlates positively with ER expression in breast cancer tissue microarray:

The PHLDA 1 expression was exclusively cytoplasmic in whole sections of normal and tumorous breast tissues. Of 114 analyzable tumors in the TMA, 90 (80%) were interpretable for PHLDA1 staining. The distribution of staining was as follows: no staining (n=59; 65.6%); mild staining (n=20; 22.2%); moderate staining (n=10; 11.1%) and strong staining (n=1; 1.1%). PHLDA1 expression was significantly associated with only ER expression (p=0.012). We did not observe a significant correlation with any of the remaining clinicopathological markers which included tumor size, type, grade, nodal status, and expression of HER2, PR and FOXA 1.

### Discussion

The chemical genetic approach for the identification of direct substrates of kinases is highly versatile and has been applied to over 40 kinases to date. A single mutation of gatekeeper residue to a glycine or an alanine for most kinases renders them sensitive to orthogonal ATP analogs and PP1-derived inhibitors. In the case of Aurora kinases, a novel mutation was identified, which is required along with the gatekeeper mutation to render them amenable to the chemical genetic approach. This technique revealed PHLDA1 as a novel substrate of Aurora A in breast cancer cells.

PHLDA1 has been shown to be both pro-apoptotic and anti-apoptotic depending on the cell line used and the conditions studied. It was initially identified in T cell hybridoma, where it mediates apoptosis by inducing Fas expression, and was thus named T cell death-associated gene 51 (TDAG51). However, other studies revealed no role of PHLDA1 in T cells apoptosis both in cells and in vivo. In IGFR-NIH3T3 cells, PHLDA1 is a critical mediator of the anti-apoptotic effect of IGF1. Similarly, PHLDA1 is highly expressed in pancreatic tumors, which are resistant to apoptosis and chemotherapeutic agents.

PHLDA1 regulates apoptosis in vascular endothelial cells triggered by homocysteine (Hossain, et al. (2003) J. Biol. Chem. 278, 30317-30327). In neuronal cells, PHLDA1 enhances cell death, but without Fas induction (Gomes, et al, (1999) J. Neurochem. 73, 612-622). Down-regulation of PHLDA 1 expression is associated with the progression of malignant melanomas (Neef, et al, (2002) Cancer Res. 62, 5920-5929).

Our study confirmed the prior reports of PHLDA1 transcripts being downregulated in primary breast tumors (Nagai, et al, (2007) Breast Cancer Res. Treat. 106, 49-56). In almost 66% of the cases in the current study, there were no detectable levels of the protein. This is similar to the 72% down-regulation of PHLDA1 proteins that has been previously reported (Nagai, et al, (2007) Breast Cancer Res. Treat. 106, 49-56). The differences in association with between PHLDA1 expression with ER in the current study and that of Nagai et al could be at least in part due to different methods used for analysis (IHC versus ligand-binding assay). PHLDA1 expression does not correlate with patient age, tumor type, tumor size or nodal status. In spite of this, down-regulation of PHLDA 1 protein was a strong predictor of poor prognosis for breast cancer patients with loss of the protein predicting for adverse prognosis (Nagai, et al, (2007) Breast Cancer Res. Treat. 106, 49-56). These results provide ample evidence that reduced PHLDA1 expression is important in breast cancer progression and could serve as useful prognostic marker of disease outcome.

We show that PHLDA1 protein levels are negatively regulated by Aurora A by direct phosphorylation at Ser98 in breast cancer cells. Our results further revealed that PHLDA1 is not a mitotic target of Aurora A. PHLDA 1 and Aurora A have never been associated before. This is also the first study that shows PHLDA1 protein level is regulated by a post-translational modification. PHLDA1 also negatively affects Aurora A protein levels, thereby engaging in a negative feedback loop. PHLDA 1 overexpression inhibits cell transformation and chemotaxis, thereby revealing PHLDA 1 degradation as a key mechanism by which Aurora A promotes breast malignancy. Thus, not surprisingly, PHLDA1 upregulation acts synergistically with Aurora A inhibition in promoting cell death.

### CONCLUSION

PHLDA1 downregulation and Aurora A upregulation are strong predictors of poor prognosis for breast cancer patients. However, they have been not analyzed together. Thus, our finding showing Aurora A and PHLDA 1 in a negative feedback loop highlights the relevance of this study. We further show that PHLDA1 is a strong inhibitor of cell motility, proliferation and transformation in breast cancer cells, suggesting PHLDA1 overexpression may be an alternative way to modulate Aurora A deregulation in breast cancer. Analysis of PHLDA1 and Aurora A levels could supplement standard staging information in primary biopsy samples. Results from these studies can facilitate the development of combination therapies using both Aurora A and PHLDA1-targeted drugs. Finally, a novel mutation was identified in this study, which is required (along with the gatekeeper mutation) to render Aurora kinases amenable to the chemical genetic approach. We expect that this new mutation will give researchers a versatile tool for creating allele-specific mutants of kinases that hitherto have not been amenable to chemical genetic methodology.

### Example 2

Identification of LIMK2 as a Positive Regulator and Effector of Aurora A Kinase.

### Materials

Antibodies for Aurora A (H-130), α-tubulin (B-7), actin (C-2), histone H3 (Ser10)-R and LIMK2 (H-78) were purchased from Santa Cruz Biotech (Santa Cruz, Calif., USA). Matrigel was obtained from BD Biosciences (Bedford, Mass., USA).

### Cell Culture

HCT116, MDA-MB-231, HEK-293T, PC3, and PaCa2 cells were purchased from ATCC (Manassas, Va., USA). Except for HEK-293 cells, all other cell lines were cultured in RPMI with 10% FBS supplemented with 2 mM glutamine and antibiotics (Penicillin G/streptomycin). HEK-293T cells were cultured in DMEM with 10% FBS supplemented with 2 mM glutamine and antibiotics (Penicillin G/streptomycin).

### Expression Plasmids and Constructs

HA-tagged LIMK2 was cloned into VIP3 mammalian vector and pTAT/pTAT-HA vector at BamHI and XhoI sites. HA-tagged LIMK2 mutants were generated using overlapping PCR.

Expression and Purification of TPX2, Aurora A and LIMK2 Aurora A and TPX2 were prepared using the baculovirus Bac-to-Bac expression system according to manufacturer's instructions (Invitrogen). Protein concentration was determined using Bradford assay, and the protein purity was assessed by Western blotting using 6-His antibody. 6-His-LIMK2 was expressed in E. coli and purified as described before Sun, et al, (2008a) Mol. Biol. Cell 19, 3052-3069).

### Transfection and Retroviral Infection

For generating stable cell lines, Aurora A and LIMK2 plasmids were transiently transfected using calcium phosphate into Phoenix cells. The retroviruses were harvested and used to infect MDA cells as reported previously (Shah and Shokat, (2002) Chem. Biol. 9, 35-47).

### In Vitro Kinase Assays

For in vitro labeling, 2 μg of 6-His-tagged recombinant protein (such as LIMK2 or cofilin) was added into kinase buffer along with Aurora A/TPX2 complex and 0.5 μCi of [γ-32P] ATP. Reactions were terminated by adding SDS sample buffer, separated by SDS-PAGE gel and then transferred to PVDF membrane and exposed to Biomax MS film.

LIMK2 and Aurora A shRNA

LIMK2 shRNAs were cloned into pLKO.1 TRC vector, which was a gift from David Root (Moffat, et al. (2006) Cell 124, 1283-1298). LIMK2 shRNA were designed as follows: (1) LIMK2-shRNA1-forward oligo 5′-CCGGCCAACTGGTA CTATGAGAACTCGAGTTCT CATAGTACCAGTTGGTTTTTG-3′ (SEQ ID NO: 20) and reverse oligo 5′-AAT TCAAAAACCAACTGGTACTATGAGAACT CGAGTTCTCATAGTACCAGTTGG-3′ (SEQ ID NO: 21); (2) LIMK2-shRNA2-forward oligo 5′-CCGGGCTATTCACAGCAGATCTTCTCGAGAAGATC TGCTGTGAATAGCTTTTTG-3′(SEQ ID NO: 22) and reverse oligo 5′-AATTCAAAAAGCTATTCACAGCAGATCTTCTCGAGAAGATCT GCTGTGAATAGC-3′ (SEQ ID NO: 23); (3) LIMK2-shRNA3-forward oligo 5′-CCG GCCTGCTGACAGAGTACATTCTCGAGAATGTACTCTGTCAGCAGGTTTTTG-3′ SEQ ID NO: 24) and reverse oligo 5′-AATTCAAAAACCTGCTGACAGAGTACA TTCTCGAGAATGTACTCT GTC AGCAGG-3′ (SEQ ID NO: 25). Aurora A shRNA were generated in our previous study (Johnson et al., (2011) J. Cell Science 124 (6) 2711-2722). Control shRNA (scrambled shRNA) and LIMK2 shRNA Aurora A were transfected to MDA cells using Lipofectamine following manufacturer instructions. Alternatively, AA shRNA and LIMK2 lentiviruses were generated and used for infecting MDA cells. LIMK2-ablated MDA and LIMK2-ablated-AA-MDA stable cells were generated following puromycin selection.

### 1-NM-PP 1 and MLN 8237-Mediated Inhibition of AA

AA-MDA and AA-as7 MDA cells were treated with either DMSO or 1-NM-PP1 (250 nM) for 12 h, followed by cell lysis. For MLN8237 experiment, MDA cells were treated with 1 μM MLN8237 for 12 h.

### Soft Agar Colony Formation

MDA, AA-MDA, LIMK2-ablated MDA and LIMK2-ablated AA-MDA cells were plated in RPMI (103, 104 and 105 cells per dish in triplicate), 0.3% agar and 10% calf serum in six-well plates as reported before (Shah and Shokat (2002) Chem. Biol. 9, 35-47). Transformed colonies were counted after three weeks.

### Cell Synchronization

MDA, LIMK2-MDA or LIMK2-ablated MDA cells were treated with 2.5 mM thymidine for 16 hours, released for 8 hours, and then treated with thymidine for an additional 16 hours. After two washes with phosphate-buffered saline (PBS), cells were cultured for different times as indicated in the experiment and harvested.

### Western Blotting

Cells were harvested and lysed in modified RIPA buffer, supplemented with protease inhibitors. Equal amounts of cell extracts were then used to conduct western blot.

### Ubiquitination Assay

MDA cells were co-transfected with LIMK2 or Aurora A shRNA along with 6-His-Ubiquitin. After 36 h, MG132 (Sigma) was added at 10 μM final concentration for additional 12 h. Cells were then harvested, and ubiquinated proteins were isolated using Ni-NTA beads. The proteins were separated by SDS-PAGE and analyzed using AA and LIMK2 antibodies.

### Chemotaxis Assay

MDA, AA-MDA, LIMK2-ablated MDA and LIMK2-ablated AA-MDA cells were serum starved in serum-free RPMI for 12 h and isolated by limited trypsin digestion. Cell migration was determined in Boyden chambers as reported before (Shah and Vincent (2005) Mol. Biol. Cell 16, 5418-5432.). The assays were performed in triplicate, four independent times. To allow for comparison between multiple

assays, the data were normalized, and expressed as a percentage of the number of cells present on the membrane.

### MTT Assay

Cells were seeded in 96-well plate at 1,500 cells per 100 μL per well and cultured for 24, 48, and 72 h. MTT was conducted as reported before (Sun, et al., (2009) Mol. Biol. Cell 20, 4611-4619). Experiments were repeated three times in quadruplicate wells to ensure the reproducibility of results.

### Immunofluorescence

MDA, LIMK2-MDA, LIMK2-ablated MDA or LIMK2-ablated AA-MDA cells were grown on poly-lysine coated coverslips for 24 h, fixed with 4% formaldehyde in PBS for 15 min at RT, and then washed 3 times with PBS. The cells were permeabilized with 0.2% Triton in PBS for 5 min, washed twice with PBS, and blocked in 5% BSA/PBS for 2 h at 25° C. Cells were labeled with primary antibodies (Aurora A, α-tubulin, phospho-histone H3 or LIMK2 for 3 h in 1% BSA/PBS, followed by incubation with fluorescein isothiocyanate or Texas Red conjugated secondary antibody. Cells were counterstained with prolong antifade and visualized using a Nikon TE2000 inverted confocal microscope (Nikon, Tokyo, Japan) with a Radiance 2100 MP Rainbow Laser (Bio-Rad Laboratories).

### In Vivo Xenograft in Nude Mice

All the animal experiments were done in accordance with institutional guidelines of the Purdue Animal Care and Use Committee. Female athymic nude mice 4-5 weeks of age were obtained from Harlan Laboratories (Indianapolis). Five to six weeks old nude mice weighing 18-22 g were anesthetized and inoculated with MDA (5×106/mouse), AA-MDA cells (5×106/mouse) and LIMK2-ablated AA-MDA cells (5×106/mouse). AA-MDA cells were implanted on the right shoulders and LIMK2-ablated AA-MDA cells were implanted on the left shoulders of the same four mice. To compare the tumorigenic potential of MDA and AA-MDA cells, MDA cells were planted on the right shoulder, and AA-MDA cells were planted on the left shoulder of athymic nude mice in a different set of experiments. Prior to implantation, cells were harvested via trypsinization, washed twice with PBS and resuspended in 200 μl RPMI and matrigel in 1:1 ratio. Tumor growth was monitored and measured every two days in two perpendicular directions using a caliper (body weights were monitored on the same schedule), and the volumes of the tumors were calculated as 0.5×L×W2, where L is the longest axis and W is the axis perpendicular to L in millimeters. All mice were sacrificed 34 days following inoculation, tumors were dissected and their sizes were compared. Mice bearing tumors did not display any weight loss compared to control mice at the time of sacrifice.

### Statistical Analysis

Bar graphs results are plotted as the average±SEM. Significance was evaluated using Student's t test analysis and is displayed as follows: *p>0.05, **p>0.01, ***p>0.001.

### Results

### LIMK2 is a Novel Aurora A Substrate:

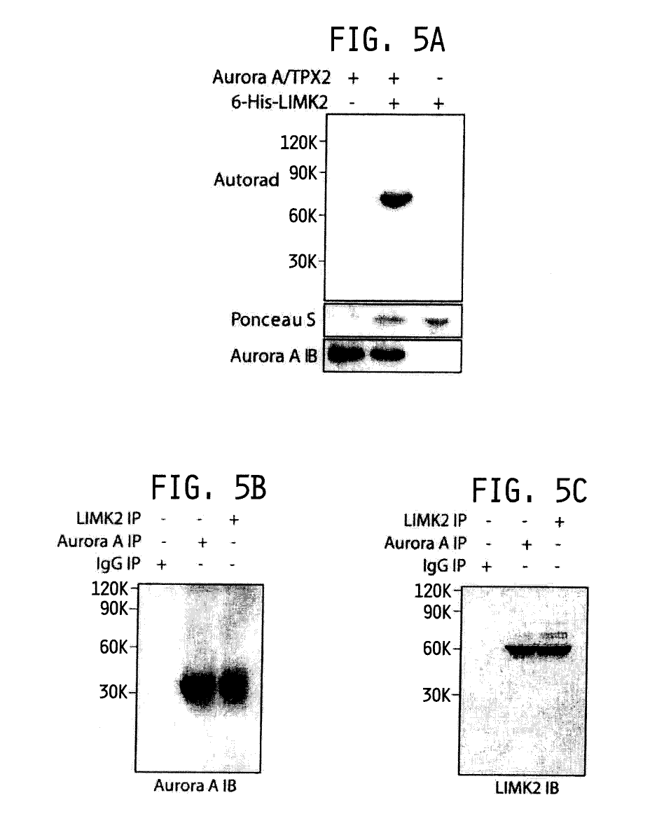

LIMK2 phosphorylation was determined using recombinant Aurora A/TPX2 and 6-His-LIMK2 in an in vitro kinase assay. TPX2 is an activator of Aurora A. Our results show that Aurora A directly phosphorylates LIMK2 (FIG. 5A, lane 2). We next investigated whether Aurora A and LIMK2 associate in MDA cells. LIMK2 immune complex was isolated and Aurora A binding analyzed, which revealed that Aurora A associates with LIMK2 (FIG. 5B, lane 3). Similar results were obtained when Aurora A immune complex was isolated and LIMK2 binding analyzed (FIG. 5C, lane 2). Accordingly, the results demonstrate that LIMK2 is an Aurora A substrate that is directly phosphorylated by Aurora A.

### Aurora A Regulates LIMK2 Levels Using its Kinase Activity:

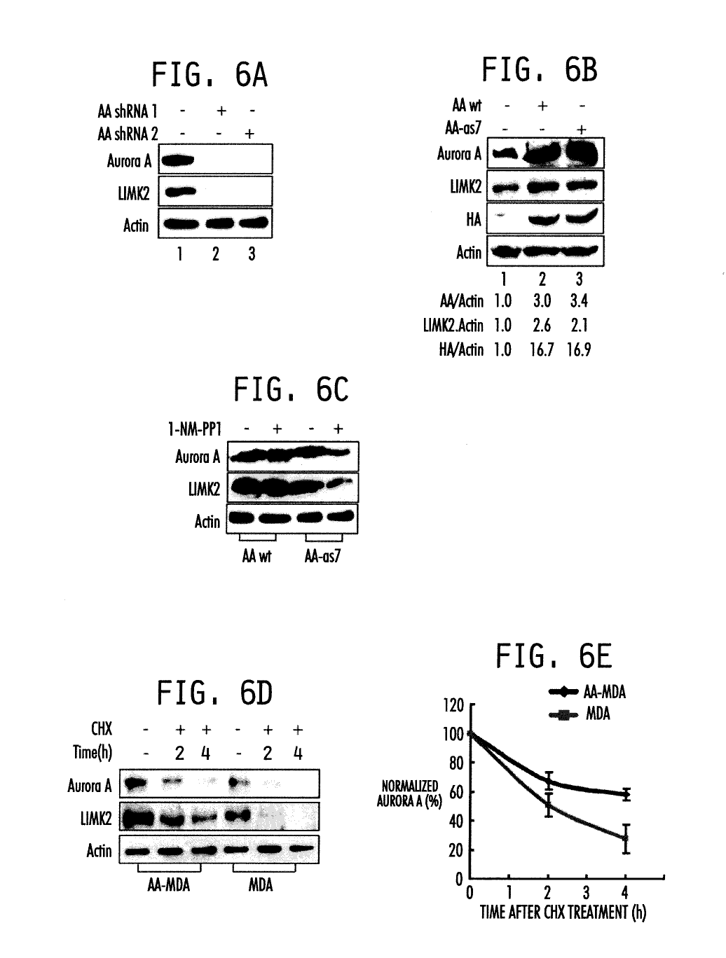

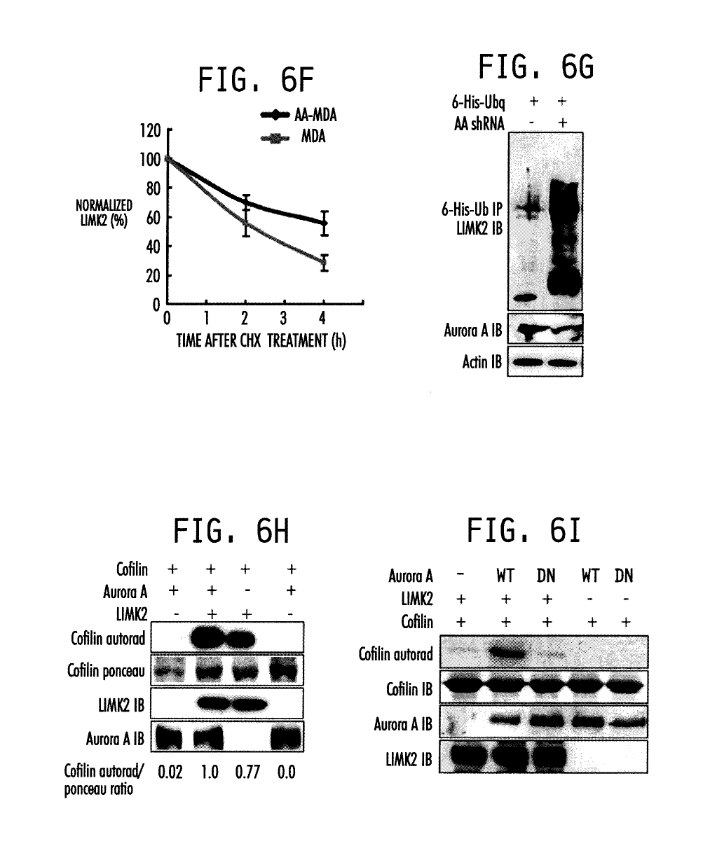

Aurora A-mediated phosphorylation of its substrates often promotes their degradation (e.g. p53, PHLDA1) or stabilization (ASAP1). Therefore, we investigated whether Aurora A exerts control over LIMK2 level. Aurora A was transiently depleted in MDA cells using two different shRNAs, causing concomitant decrease in LIMK2 level (FIG. 6A). Wild-type and mutant AA (AA-as7) were stably overexpressed in MDA cells (AA-MDA and AA-as7-MDA cells respectively), leading to considerable increase in LIMK2 levels (FIG. 6B), thereby confirming that Aurora A positively regulates LIMK2 levels. Aurora A can regulate its substrates using either its kinase activity or scaffolding function. Aurora A regulates p53 levels by phosphorylation, but regulates ASAP levels through the scaffolding function. Therefore, to dissect the mechanism by which Aurora A regulates LIMK2, AA-MDA and AA-as7-MDA cells were treated with 1-NM-PP 1 for 12 h and LIMK2 expression analyzed. 1-NM-PP 1 is a highly selective and potent inhibitor of Aurora A-as7 kinase, and does not inhibit wild-type Aurora A even at higher concentrations. 1-NM-PP1 treatment caused significant decrease in LIMK2 level in AA-as7-MDA cells, but not in wt AA-MDA cells (FIG. 6C). This result demonstrates that Aurora A upregulates LIMK2 levels using its kinase activity, and not via scaffolding interactions.

### Aurora A Inhibits LIMK2 Degradation:

The involvement of Aurora A's kinase function in positively regulating LIMK2 suggested that Aurora A may inhibit degradation of LIMK2 via phosphorylation. Therefore, LIMK2 protein degradation profile was examined in MDA and AAMDA cells using cycloheximide. Since Aurora A has a half-life of ˜2 h, 2 h and 4 h time-point was selected. Aurora A overexpression reduced LIMK2 degradation (FIG. 6D-F), suggesting that it regulates LIMK2 level by inhibiting its degradation. This study revealed that the half-life of LIMK2 was ˜2 h (FIG. 6F). LIMK2 degradation could be mediated by ubiquitin or non-ubiquitin pathways.

6-His-Ubiquitin was transfected in MDA and Aurora A-depleted MDA cells, and LIMK2 ubiquitination analyzed. Aurora A depletion led to increased LIMK2 ubiquitination (FIG. 6G), thereby demonstrating that Aurora A stabilizes LIMK2 level by inhibiting its ubiquitination.

### Aurora A-Mediated Phosphorylation Increases LIMK2 Kinase Activity:

After confirming Aurora A-mediated stability of LIMK2 protein, we sought to determine whether Aurora A regulated LIMK2 kinase activity. Cofilin, a well characterized substrate of LIMK2, was used to evaluate LIMK2 kinase activity. Aurora A does not phosphorylate cofilin (FIG. 6H, lanes 1 and 4). LIMK2 efficiently phosphorylated cofilin as expected (lane 3). Importantly, LIMK2 preincubated with Aurora A showed increased kinase activity (lane 2), confirming that Aurora A also boosts the kinase activity of LIMK2. Aurora A can increase LIMK2 activity either by phosphorylation or by association. Therefore, LIMK2 kinase activity was tested by pre-incubating it with either wt or dominant negative (D274A) Aurora A. While wt Aurora A increased LIMK2 kinase activity significantly (as measured by increased cofilin phosphorylation, FIG. 1, compare lanes 1 and 2), dominant negative Aurora A had no effect on LIMK2 activity (FIG. 6I, lane 3). This results shows that the kinase activity of LIMK2 is predominantly regulated by Aurora A-mediated phosphorylation, and not by physical association with LIMK2.

### LIMK2 and Aurora A are Involved in a Positive Feedback Loop:

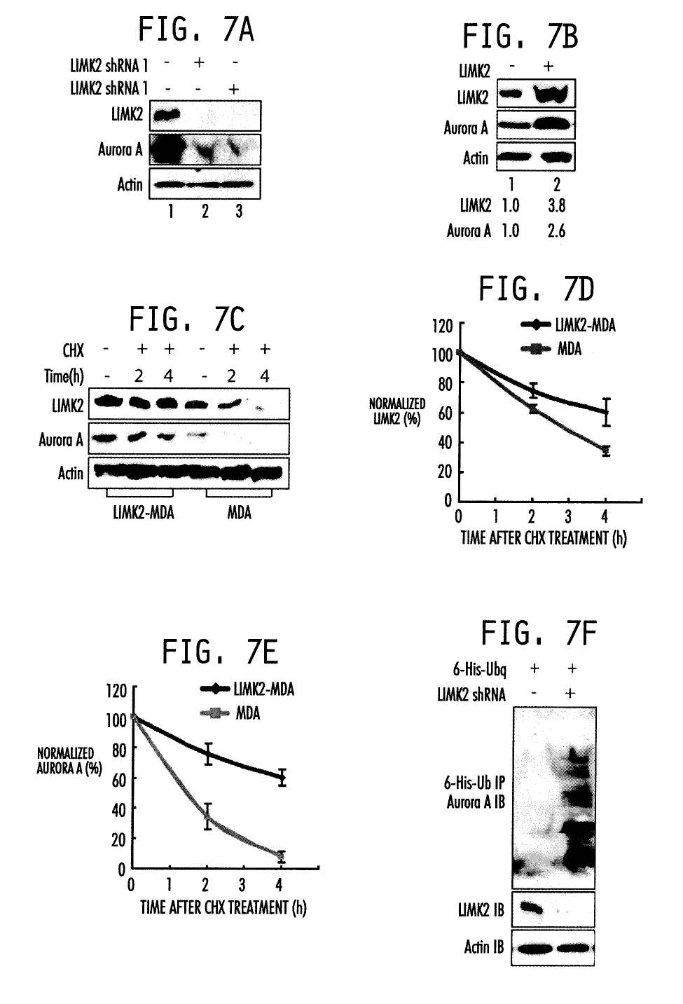

Several Aurora A substrates are known to regulate Aurora A activity/expression by a feedback mechanism. Aurora A phosphorylates FAF1 at 5289 and 5291, which in turn degrades Aurora A. Similarly, PP1 inhibits Aurora A kinase activity upon phosphorylation by Aurora A. We previously have shown that the Aurora A substrate PHLDA1 negatively regulates Aurora A in a feedback loop (see Example 1). LIMK2 shRNAs were generated and used to reduce LIMK2 levels in cells, which decreased Aurora A level significantly, suggesting the existence of a positive feedback loop between the two proteins (FIG. 7A). LIMK2-overexpressing stable MDA cells (LIMK2-MDA) were generated and Aurora A levels analyzed. LIMK2 overexpression increased Aurora A levels, confirming that LIMK2 positively regulates Aurora A (FIG. 7B).

Since LIMK2 can also increase Aurora A levels by inhibiting its degradation, Aurora A and LIMK2 levels were analyzed in cycloheximide-treated MDA and LIMK2-MDA cells. LIMK2 overexpression considerably reduced Aurora A degradation (FIG. 7C-E), suggesting that LIMK2 stabilizes Aurora A protein levels, presumably by inhibiting its ubiquitination. To test this possibility, 6-His-Ubiquitin was transfected in MDA cells and LIMK2-depleted MDA cells and Aurora A ubiquitination analyzed using 6-His antibody. LIMK2 depletion indeed increased ubiquitinated Aurora A (FIG. 7F, lane 2). These results show that Aurora A and LIMK2 positively regulate each other by stabilizing each other's protein levels.

LIMK2 does not Phosphorylate Aurora A:

Since LIMK2 is a kinase, we examined whether it phosphorylates Aurora A leading to its stabilization. Recombinant 6-His-tagged Aurora A showed negligible kinase activity in vitro in the absence of TPX2, suggesting that it could be used as a putative substrate for LIMK2. However, when LIMK2 was incubated with Aurora A (without TPX2), Aurora A did not get phosphorylated, although LIMK2 efficiently phosphorylated cofilin, which was used as a positive control. This result suggested that unlike Aurora A, which stabilizes LIMK2 via phosphorylation, LIMK2 stabilizes Aurora A by protein-protein interactions.

To confirm this finding, wt LIMK2 and kinase-inactive LIMK2 (K339M)-expressing stable MDA cell lines were generated. Interestingly, both wt and kinase-inactive LIMK2 caused an increase in Aurora A level, although, wt LIMK2 increased it more. This result suggests that LIMK2 stabilizes Aurora A using both its scaffolding interactions and kinase function. However, Aurora A is not a direct substrate of LIMK2. It is likely that LIMK2 stabilizes Aurora A indirectly by phosphorylating some other targets.

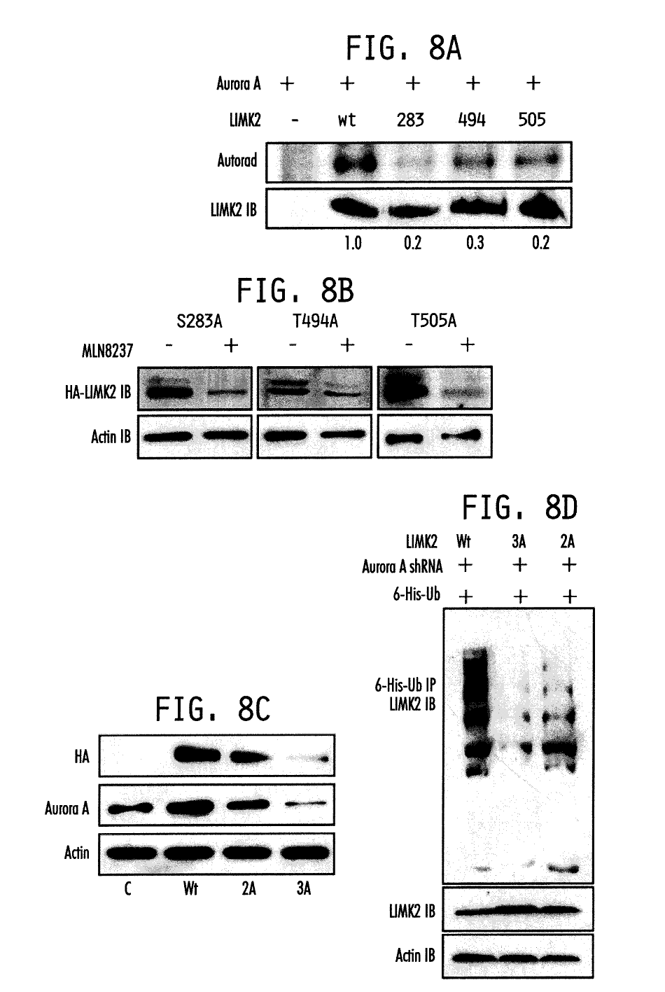

Aurora A inhibits LIMK2 degradation by directly phosphorylating it at Ser283, Thr494 and Thr505: Since Aurora A regulates LIMK2 by phosphorylation (FIG. 6A), we set to identify Aurora A-mediated phosphorylation sites on LIMK2. Aurora A preferentially phosphorylates R/K/N-R-X-S/T-B, where B denotes any hydrophobic residue except Pro (Ferrari et al. (2005) Biochem. J. 390, 293-302). This preference revealed 5283, T494 and T505 as putative Aurora A phosphorylation sites on LIMK2. S283A, T494A and T505A LIMK2 alleles were generated and their phosphorylation analyzed in vitro. Aurora A phosphorylates LIMK2 at all these sites (FIG. 8A). To investigate whether S283, T494, and T505 residues on LIMK2 are phosphorylated in cells, a gel shift assay was performed by transfecting LIMK2 phosphorylation-resistant single mutants in MDA cells. After 30 h, these cells were treated with MLN8237 for an additional 12 h Inhibition of Aurora A decreased both phosphorylation level (top band) and overall protein level of LIMK2 mutants, suggesting that these three sites are phosphorylated by Aurora A in cells (FIG. 8B). This result also suggested that phosphorylation of all three sites (S283, T494, and T505) by Aurora A contributes to LIMK2 stability.

To confirm these findings, HA-tagged LIMK2 double mutant (S283A, T494A) and HAtagged LIMK2 triple mutant (S283A, T494A, T505A) were generated and transfected in MDA cells. HA-tagged wt LIMK2 was used as a control. After 24 h, Aurora A was inhibited using MLN8237 for an additional 12 h, and LIMK2 levels analyzed using HA antibody. Wild type LIMK2 showed highest expression levels, followed by LIMK2 double mutant. LIMK2 triple mutant showed least expression. Importantly, Aurora A inhibition decreased phospho-levels and protein levels of both wt LIMK2 and (S283A, T494A) LIMK2, but the protein and phospho-levels of LIMK2 triple mutant remained unchanged. Together, these findings demonstrate that Aurora A modulates LIMK2 stability by phosphorylating S283, T494, and T505 sites in cells.

Since LIMK2 levels modulate Aurora A levels in a feedback loop, we compared LIMK2 (HAtagged) and Aurora A levels in MDA, wt LIMK2-MDA, (S283A, T494A) LIMK2-MDA and (S283A, T494A, T505A) LIMK2-expressing MDA cells. While wt LIMK2 expressing MDA cells showed robust expression of Aurora A and LIMK2, (S283A, T494A), LIMK2-MDA cells showed reduced, and (S283A, T494A, T505A) LIMK2-MDA cells negligible, expression (FIG. 8C, top panel). These findings confirm that Aurora A stabilizes LIMK2 levels by direct phosphorylation at S283, T494 and T505, which in turn promotes Aurora A stabilization in a positive feedback loop (FIG. 8C, middle panel). This result is consistent with our previous data whereby inhibition of Aurora A kinase activity reduces LIMK2 protein level, in turn reducing Aurora A level.

We next analyzed ubiquitination of wt and triple LIMK2 mutants in MDA cells, which showed robust ubiquitination of LIMK2 triple mutant, but not of wt LIMK2 (FIG. 8D), suggesting that Aurora A-mediated phosphorylation of LIMK2 stabilizes its protein levels.

### Aurora A Increases LIMK2 Kinase Activity by Phosphorylating T505:

Previous studies have shown that LIMK2 phosphorylation at T505 by ROCK and MRCK-alpha kinases increases its kinase activity (Sumiet al (2001b) J. Biol. Chem. 276, 23092-23096). Since we observed T505 phosphorylation by Aurora A, it provides the mechanism by which Aurora A increases LIMK2 kinase activity. However, to confirm that the increased activity was only due to T505 phosphorylation and not due to phosphorylation at other sites, we measured Aurora A-mediated activation of wt LIMK2, (S283A) LIMK2, (T494A) LIMK2 and (T505A) LIMK2 in an in vitro kinase assay using cofilin as the substrate. As expected, Aurora A activated wt LIMK2, (S283A) LIMK2 and (T494A) LIMK2, but not (T505A) LIMK2 mutant. Furthermore, Aurora A activated (S283A, T494A)-LIMK2 double mutant, but not (S283A, T494A, T505A)-LIMK2 triple mutant, thereby demonstrating that Aurora A activates LIMK2 by phosphorylating T505.

### Aurora A Promotes Cytoplasmic Localization of LIMK2:

Previously, PKC has been shown to phosphorylate LIMK2 at S283 and T494 sites, which reduces its nuclear import (Goyal et al., (2006) J. Biol. Chem.

281, 25223-25230). We also observed nuclear localization of LIMK2 upon Aurora A inhibition. These results were further confirmed in Aurora A-ablated MDA cells. Specifically, scrambled or Aurora A shRNA were transfected in MDA cells, and after 30 h, LIMK2 localization was analyzed, which showed nuclear localization of LIMK2, suggesting that similar to PKC, Aurora A-mediated LIMK2 phosphorylation also localizes it in the cytoplasm. 1 μM PKC412 (12 h) treatment was used as the control.

### Aurora A Associates LIMK2 via LIM Domains:

Since Aurora A and LIMK2 showed strong association (FIGS. 5B and 5C), we examined the LIMK2 domain responsible for Aurora A association. LIMK2 contains two LIM domains (12-129), a PDZ domain (152-236), an S/P domain (240-328) and a kinase domain (331-601) (Scott and Olson (2007) J. Mol. Med. 85, 555-568). Therefore, different HA-tagged truncated LIMK2 mutants were generated containing either two LIM domains (1-145), LIM and PDZ domains (1-298) or predominantly kinase domain (298-638). These mutants were transiently expressed in MDA cells, isolated and analyzed for Aurora A binding. Our results revealed that LIMK2 binds Aurora A via its LIM domains (1-145).

### Aurora A-LIMK2 Association is Partially Responsible for LIMK2 Stabilization:

We next investigated whether association of LIM domains of LIMK2 is sufficient for Aurora A stabilization. Aurora A levels were examined in MDA cells, LIMK2-MDA cells and (1-145) LIMK2 mutant-expressing MDA cells. We generated HA-tagged (1-145) LIMK2, (1-298) LIMK2 and (298-638) LIMK2 to investigate Aurora A-LIMK2 association. LIMK2 associates with Aurora A using its LIM domains (1-145). HA-tagged wild-type LIMK2, (1-145) LIMK2, (1-298) LIMK2 and (298-638) LIMK2 mutants were transfected in MDA cells. After 30 h, cells were lysed, HA-tagged LIMK2 proteins were isolated and probed for Aurora A binding. HA-tagged wild-type LIMK2 and (1-145) LIMK2 mutant were transfected in MDA cells. After 30 h, cells were lysed, HA-tagged LIMK2 proteins were isolated and probed for Aurora A binding. While expression of full length LIMK2 robustly increased Aurora A levels, (1-145) LIMK2 expression partially increased it. This result suggests that although LIMK2-Aurora A association increases Aurora A levels, LIMK2 kinase activity is also an essential contributor to Aurora A stability. This result is consistent with our previous finding that showed LIMK2 stabilizes Aurora A using both its scaffolding interactions and kinase function.

### LIMK2 is not a Mitotic Target of Aurora A:

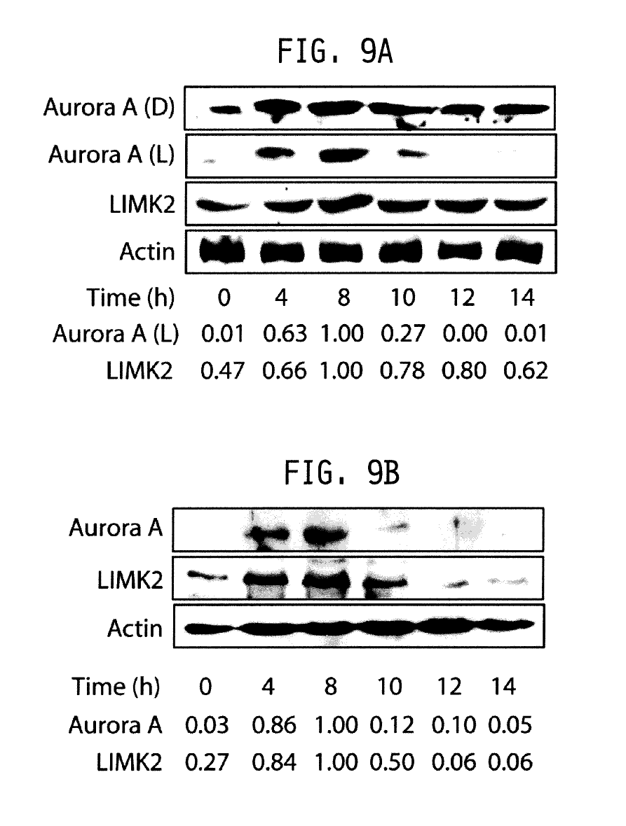

To investigate whether LIMK2 plays a role in mitosis similar to Aurora A's, G1/S arrested MDA cells were released for varying periods, and LIMK2 and Aurora A levels analyzed. LIMK2 was expressed throughout the cell cycle, whereas Aurora A expression markedly increased during mitosis (FIG. 9A), suggesting that LIMK2 expression is not cell cycle regulated. Still, positive correlation was observed between Aurora A and LIMK2 levels, and increased Aurora A levels were closely linked with increased LIMK2 levels (FIG. 9A). We also examined the levels of LIMK2 and Aurora A in HCT116 cells, which shows tight cell cycle regulation. The expression of LIMK2 was strongly correlated with Aurora A levels, and markedly increased after 6 h and 8 h, which were coupled with maximal Aurora A expression (FIG. 9B). Taken together, our data reveal that Aurora A regulates a significant population of LIMK2 in cancer cells. FACS analysis was conducted to examine a potential role of LIMK2 in mitosis using unsynchronized MDA, LIMK2-MDA stable cells and LIMK2-ablated MDA (stable) cells. While MDA cells showed no aneuploidy, LIMK2-MDA and LIMK2-ablated

cells showed some aneuploidy.

To probe the mechanism further, histone H3 (Ser10) phosphorylation (a mitotic marker) was analyzed in synchronized MDA, LIMK2-ablated and LIMK2-MDA cells. Both MDA and LIMK2-MDA cells showed similar percentage of phospho-histone H3-positive cells at different times upon release from thymidine block, however, LIMK2 ablated MDA cells showed slightly less phospho-histone H3-positive cells. Importantly, Aurora A levels are reduced (but not depleted) in LIMK2-ablated stable MDA cells, suggesting that reduced Aurora A levels may be sufficient to carry out normal mitotic functions in MDA cells. Similarly, LIMK2-overexpressing MDA cells show increased Aurora A levels, which is well tolerated in MDA cells as observed with AA-MDA and AA-as7-MDA cells.

Since Aurora A plays a vital role in mitotic spindle formation, we further examined the consequences of LIMK2 overexpression/ablation in mitotic spindle assembly. MDA, LIMK2-MDA, LIMK2-ablated MDA and LIMK2-ablated AA-MDA cells arrested at G1/S were released for varying periods, and mitotic spindle analyzed. LIMK2 overexpression did not affect mitotic spindle assembly in majority of the cells (˜95%). In contrast, ˜40% of LIMK2-ablated cells showed defect in mitotic spindle. The remaining 60% cells were normal. Since LIMK2-ablated MDA cells comprise pooled population, varying LIMK2 levels are expected in different populations. We speculate that the cells with severely reduced LIMK2 levels show defective mitotic spindle and multiple nuclei, which is consistent with Aurora A ablation. This result is also consistent with FACS data, which revealed relatively more aneuploidy in LIMK2-ablated cells than LIMK2-MDA cells. These results suggest that LIMK2 is not directly involved in mitosis; however, LIMK2 indirectly affects mitosis by regulating Aurora A levels. LIMK2-ablated AA-MDA cells showed normal mitotic spindle in vast majority of cells, which was expected as these cells contain relatively higher AA levels as compared to LIMK2-ablated MDA cells.

### LIMK2 is a Positive Regulator of Aurora A-Mediated Breast Oncogenesis:

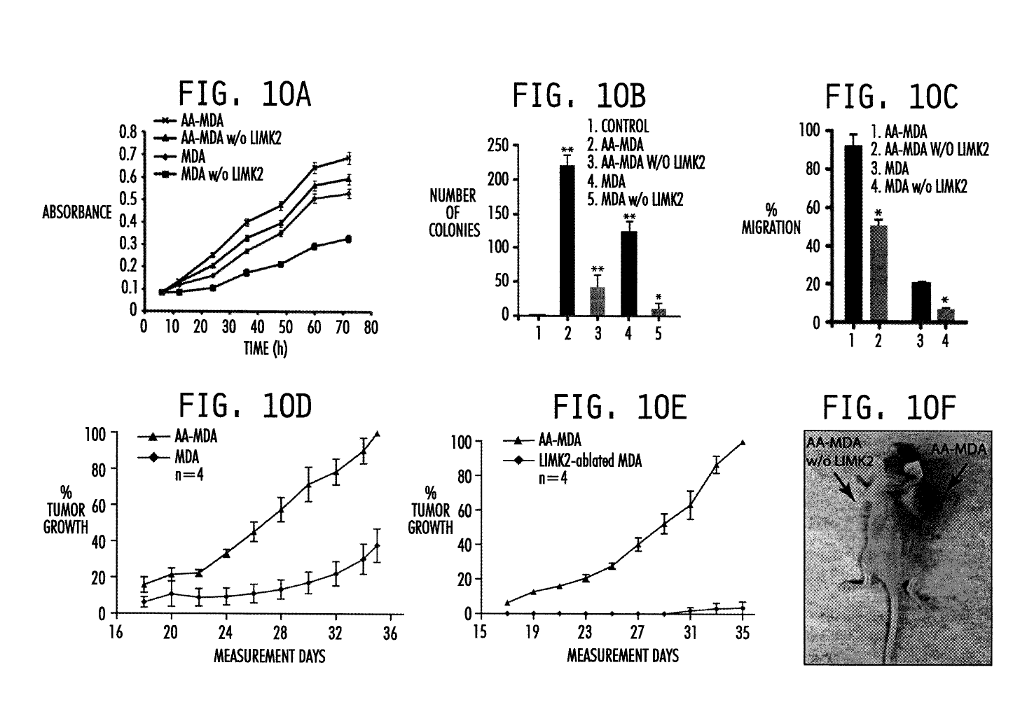

LIMK2 promotes metastasis in pancreatic cancer and fibrosarcoma, but has not been analyzed in breast malignancy. AA overexpression increased cell proliferation in MDA cells, whereas LIMK2 depletion from both MDA and AA-MDA cells reduced cell growth significantly (FIG. 10A). Previous studies have shown that LIMK2 promotes metastasis in fibrosarcoma by increasing anchorage-independent growth and cell motility, but not cell proliferation (Suyama et al., (2004 J. Gene. Med. 6, 357-363). This is the first study that shows a positive role of LIMK2 in cell proliferation.

LIMK2's effect was further evaluated in MDA and AA-MDA cells under anchorage independent conditions. Both MDA and AA-MDA cells revealed dramatic loss in colony forming ability upon LIMK2 depletion (FIG. 10B). These findings show that LIMK2 depletion inhibits cell proliferation both under attached and anchorage-independent conditions in breast cancer cells. More importantly, the striking increase in colony forming ability of MDA cells upon Aurora A overexpression (FIG. 10B, compare columns 1 and 3) is almost completely lost upon LIMK2 depletion (compare columns 2 and 4), suggesting that LIMK2 upregulation is one of the key mechanisms by which Aurora A promotes cellular transformation.

### LIMK2 is a Positive Regulator of Chemotaxis:

We next determined LIMK2's contribution in promoting chemotaxis using serum-starved MDA, AA-MDA, LIMK2-depleted MDA and LIMK2-depleted AA-MDA cells. While AA-MDA cells were highly motile, LIMK2 depletion reduced cell motility considerably (FIG. 10C). As chemotaxis plays a key role in cancer metastasis, these results underscore a critical oncogenic role of LIMK2 in breast malignancy.

### Aurora A Overexpression Promotes Tumorigenesis In Vivo:

Our cellular data strongly suggested that Aurora A-mediated oncogenic phenotypes can be reversed by LIMK2 ablation in vivo (FIG. 10A-C). Therefore, we initially determined Aurora A's contribution in inducing tumorigenesis in a mouse xenograft model. MDA and AA-MDA cells were subcutaneously inoculated on the right and left shoulders respectively in athymic nude mice, and tumors were measured every two days for a period of five weeks. AA overexpression caused robust increase in tumorigenesis in vivo (FIG. 10D).

### LIMK2 Ablation Abrogates Aurora A-Mediated Malignancy In Vivo:

We investigated whether LIMK2 ablation in MDA cells reverses tumorigenesis in mouse xenograft models. Athymic nude mice were subcutaneously inoculated with AA-MDA cells and LIMK2-depleted AA-MDA cells on the right and left shoulders respectively. The tumors became measurable 12-14 days after implantation, and were measured every two days. Mice were observed over a period of five weeks and no signs of toxicity were observed. As shown in FIGS. 10E and 10F, LIMK2 ablation prevented tumor formation in nude mice. These results confirm that LIMK2 is a key oncogenic effector of Aurora A in breast malignancy. Since Aurora A is an essential kinase, inhibition or ablation of LIMK2 appears to be a viable alternative for preventing or treating breast cancer.

### LIMK2 Upregulation by Aurora A is a Common Mechanism in Many Cancers:

Aurora A is overexpressed in many cancers. Since LIMK2 was found to be a key oncogenic regulator and effector of Aurora A in breast cancer cells, we investigated whether a similar mechanism exists in other types of cancers. Aurora A has been shown to be overexpressed in 96% of high-grade prostate intraepithelial neoplasia (PIN) and 98% of prostate cancer lesions (Lee, et al., (2006) Cancer Res. 66, 4996-5002). Aurora A ablation in PC3 cells suppresses cell growth, induces apoptosis, attenuates cell migration and inhibits the growth of human prostate cancer xenografts in nude mice (Qu et al. (2008) Cancer Gene Ther. 15, 517-525). Thus, we chose prostate cancer cells to examine if a LIMK2 and Aurora A feedback loop exists in these cells. LIMK2 has not been previously analyzed in prostate cell lines or tissues.

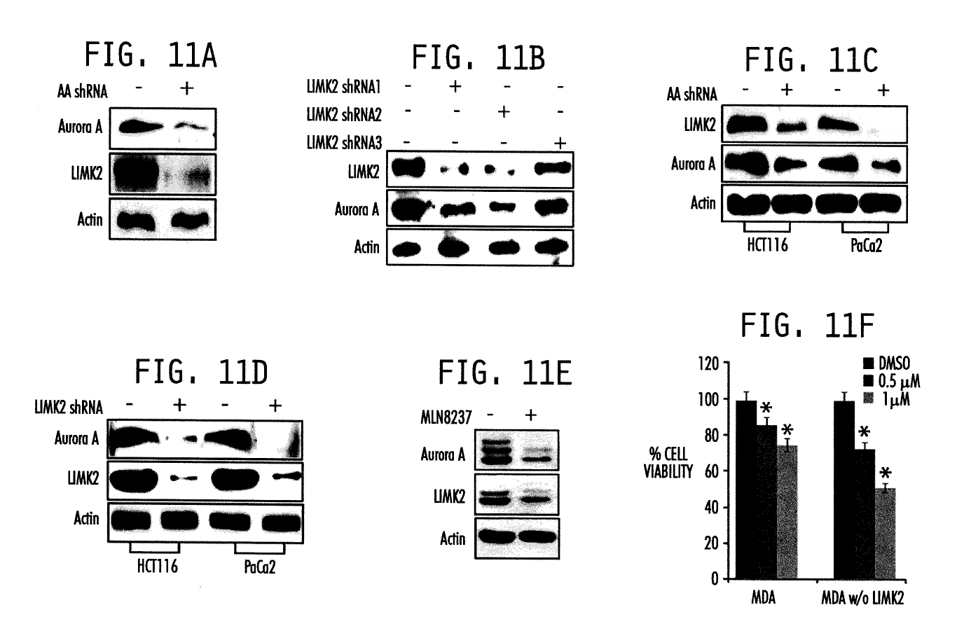

LIMK2 and Aurora A levels were examined in PC3 cells, which are highly metastatic and express high levels of Aurora A. Most importantly, Aurora A ablation in PC3 cells depleted LIMK2 levels, confirming that Aurora A positively regulates LIMK2 levels in these cells (FIG. 11A). Furthermore, similar to breast cancer cells, LIMK2 positively regulates Aurora A in prostate cancer (FIG. 11B), since LIMK2 ablation depleted Aurora A. These findings demonstrate that LIMK2 is an important effector of Aurora A-mediated oncogenesis in prostate cancer cells as well.

Aurora A and LIMK2 levels were further analyzed in pancreatic cancer (PaCa2) and colorectal cancer cells (HCT116). Both Aurora A and LIMK2 were highly expressed in these cells. Aurora A ablation in PaCa2 and HCT116 cells depleted LIMK2 (FIG. 11C).

Similarly, LIMK2 ablation depleted Aurora A, suggesting that a feedback loop involving Aurora A and LIMK2 is a common mechanism in Aurora A-induced malignancy (FIG. 11D).

### LIMK2 as a Pharmacodynamic Biomarker for Aurora A Activity:

Over a dozen Aurora A targeted drugs are in clinical trials. Due to the lack of oncogenic Aurora A targets, Aurora A autophosphorylation is used as a pharmacodynamic biomarker. Since we observed a short half life of LIMK2 (˜2 h) and rapid depletion of LIMK2 upon Aurora A ablation and inhibition, we examined whether Aurora A inhibition using a pharmacological inhibitor affects LIMK2 level. Aurora A was inhibited using MLN8237 and LIMK2 levels analyzed. As expected, Aurora A inhibition depleted LIMK2 levels, suggesting that LIMK2 levels could be used as a pharmacodynamic biomarker for investigational drugs that target Aurora A (FIG. 11E).

### LIMK2 and Aurora A Inhibition Act Synergistically in Promoting Cell Death:

LIMK2's positive role in Aurora A-mediated oncogenic pathways suggested that LIMK2 downregulation may work synergistically with Aurora A inhibition in promoting cell death. Aurora A was inhibited using MLN8237 in MDA and LIMK2-ablated-MDA cells. While ˜25% loss in cell viability was observed in MDA cells, >50% loss was observed in LIMK2-ablated-MDA cells (FIG. 11F). These findings suggest that LIMK2 inhibition/ablation may be an alternative approach for modulating Aurora A-mediated breast oncogenesis.

### Discussion

As disclosed herein two oncogenic effectors of Aurora A malignancy have been identified using a chemical genetic approach. The chemical genetic approach for the identification of direct substrates of kinases is highly versatile and has been applied to over 40 kinases to date. This technique revealed several new substrates of Aurora A in breast cancer cells including LIMK2 and PHLDA1. In normal cells, LIMK2 promotes cell cycle progression by regulating actin dynamics. LIMK2 directly phosphorylates cofilin, which inhibits its actin depolymerization activity. Previous studies have shown that LIMK2 promotes metastasis in fibrosarcoma and pancreatic cancer cells, however, the mechanism remains unknown. As disclosed herein LIMK2 is highly expressed in both breast and prostate cancer cells in an Aurora A-dependent manner. LIMK2 and Aurora A have never been associated before.

Aurora A and LIMK2 are involved in a positive synergistic feedback loop, where both potentiate each other's protein levels: inhibition/ablation of one protein depletes the other. Aurora A also regulates LIMK2's kinase activity and sub-cellular localization. Aurora A phosphorylates LIMK2 at S283, T494 and T505. Aurora A-mediated LIMK2 phosphorylation at T505 increases its kinase activity, while phosphorylation at all three sites contributes to protein stability. Sequence analysis revealed that LIMK2 contains KEN box motif (272-274), which is one of the mechanisms by which proteins are targeted for proteasomal destruction via APC/C complex. As shown herein, Aurora A stabilizes LIMK2 levels by inhibiting its ubiquitination. Aurora A also promotes cytoplasmic localization of LIMK2, presumably by phosphorylating S283 and T494. LIMK2 has been shown to be phosphorylated by ROCK at T505 and by PKC at S283 and T494. PKC-mediated LIMK2 phosphorylation reduces its nuclear retention.

As reported herein LIMK2 stabilizes Aurora A levels exploiting both protein-protein interactions and kinase activity. Domain mapping experiments demonstrated that Aurora A binds LIMK2 by binding to its LIM domains, which contributes to Aurora A stabilization to some extent. Although LIMK2 did not appear to directly phosphorylate Aurora A, it may phosphorylate additional substrates leading to Aurora A stabilization. This result is very important as it suggests that LIMK2 inhibitors are likely to be effective in abrogating Aurora A malignancy.

LIMK2 depletion in Aurora A-transformed MDA cells was discovered to completely inhibit tumor formation in nude nice, thereby revealing LIMK2 as a key oncogenic effector of Aurora A in breast malignancy. Interestingly, Limk2 null mice do not exhibit embryonic lethality or phenotypic abnormalities in postnatal growth and development, except for spermatogenesis in the testis, suggesting it is not an essential cell cycle gene. Therefore, systemic inhibition using LIMK2 inhibitors may have fewer side effects than Aurora A inhibition. Furthermore, LIMK2 depletion acts synergistically with Aurora A inhibition in promoting cell death. These findings suggest that a combination regimen comprising of sub-threshold levels of LIMK2 and Aurora A inhibitors may be a viable therapeutic approach for inhibiting breast tumorigenesis and perhaps other Aurora A-mediated malignancies.

Several Aurora A drugs are in clinical trials. Since most known Aurora A substrates are mitotic targets, Aurora A autophosphorylation is used as a pharmacodynamic biomarker. Our data show that LIMK2 is highly expressed in breast cancer and prostate cancer due to Aurora A kinase activity. Aurora A inhibition rapidly degrades LIMK2 (half-life ˜2 h), suggesting it can be used as a pharmacodynamic biomarker for Aurora A-targeted drugs. Sensitive pharmacodynamic biomarkers are essential for determining appropriate drug doses in clinical trials, thus preventing unnecessary toxicity.

Another interesting consequence of the feedback loop between Aurora A and LIMK2 was the observation that Aurora A inhibition using 1-NM-PP1 reduces Aurora A levels significantly. Since 1-NM-PP1 binds to Aurora A's active site, it is expected to have no effect on Aurora A stability. Several Aurora A inhibitors have been developed, but none of the studies have reported downregulation of Aurora A levels upon inhibition. Our data suggest that Aurora A downregulation upon inhibition is due to the loss of LIMK2-mediated suppression of Aurora A ubiquitination. Interestingly, when MLN8237 was used to inhibit Aurora A in MDA cells, it also showed reduced LIMK2 and Aurora A levels. These results highlight the relevance of the mechanism identified in this study.

Aurora A upregulation is a reliable predictor of poor prognosis in breast cancer patients. LIMK2 has been not analyzed in breast cancer. Our data show that LIMK2 is a strong activator of cell motility, proliferation and transformation in breast cancer cells and tumorigenesis in vivo, suggesting that LIMK2 inhibition/ablation may be an alternative approach to modulate Aurora A deregulation in breast cancer. Most importantly, LIMK2 and Aurora A are engaged in a positive feedback loop in several other cancer cells that were analyzed in this study, suggesting that it may be a common mechanism in Aurora A-mediated malignancy. Since Aurora A is overexpressed in various types of cancer, analysis of LIMK2 and Aurora A levels could supplement standard staging information in primary biopsy samples. Results from these studies have the potential to facilitate the development of combination therapies using both Aurora A and LIMK2-targeted drugs.

1. A kit for conducting Aurora A kinase reactions, said kit comprising
an Aurora A kinase;
microtubule-associated protein TPX2;
an Aurora A kinase substrate selected from the group consisting of PHLDA-1 and LIMK2; and
reagents for conducting a kinase reaction.

2. The kit according to claim 1 wherein the Aurora A kinase is complexed to said TPX2.

3. The kit according to claim 1 wherein the PHLDA-1 substrate comprises a peptide of SEQ ID NO: 1 or SEQ ID NO: 2 and the LIMK2 substrate comprises a peptide of SEQ ID NO: 3 or SEQ ID NO: 4.

4. The kit according to claim 3 wherein the Aurora A kinase comprises the sequence of SEQ ID NO: 5 or SEQ ID NO: 7.

5. The kit according to claim 4 wherein the kit further comprises labeled
ATP.

6. The kit according to claim 1 wherein the Aurora A kinase comprises the sequence of SEQ ID NO: 7.

7. The kit according to claim 5 wherein the kit further comprises an orthogonal ATP analog.

8. The kit according to claim 7 wherein the orthogonal ATP analog is N-6-Phenethyl ATP.

9. The kit according to claim 4 wherein the kit comprises an Aurora A kinase substrate negative control, said negative control comprising an amino acid sequence selected from SEQ ID NO: 8 and SEQ ID NO: 9.

10.-15. (canceled)

16. A method of inhibiting the proliferation of cells, said method comprising contacting cells with a composition comprising an LIMK2 inhibitor.

17. The method of claim 16 wherein said composition further comprises an Aurora A inhibitor.

18. A method for treating cancer, said method comprising
analyzing a primary biopsy sample wherein said analysis comprises
(a) measuring the concentration of PHLDA1 and/or LIMK2 in a biological sample obtained from a patient;
(b) comparing results from step (a) with control threshold values for PHLDA1 and/or LIMK2 concentrations to identify patients having tumors with relatively high levels of LIMK2 and/or low levels of PHLDA1;
administering an anti-cancer therapy selected from the group consisting of Aurora A, LIMK2 and PHLDA1-targeted drugs to said identified patients.

19. The method of claim 18 wherein said control threshold value represents the levels of PHLDA1 and/or LIMK2, in a biological sample typical for healthy individuals.

20. The method according to claim 19, wherein said cancer is breast cancer.

21. The method of claim 18 wherein, said method comprising the step of inhibiting or reducing LIMK2 activity in cancer cells.

22. The method of claim 18 wherein, said method comprising the step of enhancing the expression or the activity of PHLDA1 in cancer cells.

23. The method of claim 21 further comprising the step of administering an inhibitor of Aurora A.

24. The method of claim 21 wherein said method further comprising the step of enhancing the expression or the activity of PHLDA1 in cancer cells.
